(19) United States
(12) Patent Application Publication
(10) Pub. No.: US 2019/0183676 A1
(43) Pub. Date: Jun. 20, 2019

(54) Fluid Dispensing System and Method of Use

(71) Applicant: Eye Drop Imaging Technology, LLC, Fort Myers, FL (US)

(72) Inventors: Alexander M. Eaton, Fort Myers, FL (US); Ruixue Liu, Fort Myers, FL (US); Brian Bradley, Fort Myers, FL (US)

(21) Appl. No.: 16/326,316

(22) PCT Filed: Aug. 28, 2017
(86) PCT No.: PCT/US2017/048819

§ 371 Date: Feb. 18, 2019

Related U.S. Application Data
(60) Provisional application No. 62/415,384, filed on Oct. 31, 2016.
(60) Provisional application No. 62/383,231, filed on Sep. 2, 2016.

(51) Int. Cl. A61F 9/00; G16H 20/13; B65D 47/18

(52) U.S. Cl. CPC A61F 9/0026; B65D 47/18; G16H 20/13

(57) ABSTRACT

Platform or system of drop-dispenser devices and method for use thereof, where one device in a platform is active in that it is equipped with a data-recording/transmitting/processing/feedback unit configured to provide feedback on whether a drop is delivered to the ROI as intended, while another device is passive in that (being otherwise substantially structurally similar in configuration and operation to the active device), it does not have such a unit and is structured to simply hold, stabilize, and/or or position a liquid-drop container to aid in successful delivery of the liquid drop(s). While using the system, the user may start using the passive device and switch to the use of an active device if the results of delivery of drops to the ROI with the passive device is unsatisfactory with respect to a schedule, or success rate, or for any other reason. The use may start using the active device first and, once the period of training (with the feedback provided by the active device) is over and the habit or routine of the use of the active device without supervision is satisfactory—switch to the use of the passive device. The geometry of devices in the system is judiciously chosen to facilitate increase in success rate of as-intended drop delivery.

### CROSS-REFERENCE TO RELATED APPLICATIONS

The present application claims priority from and benefit of US Design Patent Application No. 29/615,056 filed on Aug. 25 2017; US Design Patent Application No. 29/611,244 filed on Jul. 19, 2017; US Provisional Patent Application No. 62/415,384 filed on Oct. 31, 2016, and US Provisional Patent Application No. 62/383,231 filed on Sep. 2, 2016. The disclosure of each of the above-identified patent applications is incorporated by reference herein.

### TECHNICAL FIELD

The present invention relates to systems, and methods of use, of devices that aid in the delivery of a liquid drop to a region of interest (ROI). For example, the delivery of a drop from a dispenser to an eye can be aided by the use of a device which helps to stabilize the dispenser in the correct position for ensuring successful delivery of a drop of liquid to the eye. The liquid drop delivery devices can also be structured to accommodate single-use containers of liquid. Additionally, the liquid drop delivery devices can be equipped with a feedback system (an optical system, a sensor system, etc.) for collecting and providing feedback data on whether a drop is successfully delivered to the ROI or indicating whether and when the device is being used, or (alternatively or in addition) with a delivery system to assist and/or automate the delivery process.

### BACKGROUND

The successful delivery of liquid from a squeezable container to a pre-determined region of interest (as a non-limiting example, the application of a liquid drop to a non-heterogeneous biological target) can be a difficult task. For example, applying eye drops to the eye is difficult, with many people experiencing difficulty getting each and every drop in the eye. A number of devices have been developed to aid in eye drop delivery, and they each have their own set of issues on drop delivery. For example, those devices that rest on the tissue around the eye make it difficult to hold the eye lid open to insure the drop gets into the eye, and there is a natural tendency to close the eye when it is covered.

Current devices that aid in the application of a liquid drop to a ROI also lack the ability to conform to the specific needs of the user. There is an unfulfilled need for an operational device-based platform that facilitates the successful delivery of liquid drops to the ROI based on the specific need of an individual user, where the need of the individual user may change over time.

### SUMMARY

An embodiment of the present invention provides a set of devices configured to retain a dispenser of liquid. As used herein, and unless expressly defined otherwise, the terms “set”, “kit”, “array” where applied to devices are intended to define and cover one or more devices. A device in the set includes a base with a top side and a bottom side (the base having first and second base ends); an arm extending from the top side of the base at a first end of the arm; and a holder portion with a bore formed through the holder portion and having a bore axis, the bore sized to removably retain an end of the dispenser. The holder portion is affixed to a second end of the arm to define a target region for drops emitted from a tip of the dispenser (when the dispenser is retained in the bore) in a first plane parallel to a plane that passes through the first and second base ends, the target being substantially centered at the bore axis. The device may optionally contain a unit that includes an optical system with a field-of-view (FOV). Such unit is cooperated with the arm to orient the FOV to cover the tip and the target region (when the dispenser is retained in the bore) to dispense the liquid towards the target region. In one embodiment, the base is arcuately shaped and dimensioned to fit above and in contact with a user's nose bridge; the bore axis passes through an eye of the user when the base is positioned on the user's nose bridge; and/or the bore has a cross-section (in a plane transverse to the bore axis) which is one of (i) a rectangular cross-section and (ii) as a cross-section in which the bore defines a curve having a radius of curvature (in one embodiment—a constant radius of curvature). Alternatively or in addition, at least one of a length of the arm and an inclination of the arm with respect to the base is adjustable to position the bore axis to pass through a user's eye once the base is positioned on a user's nose bridge. The device may further comprise a wing portion affixed to the arm at a point between holding portion and the base and extending from the arm along an axis that is transverse to a reference plane, which reference plane contains the bore axis and passes through the arm. In a specific embodiment, the base may be curved in a plane containing the bore axis, while a cross-section of the bore defined in a plane perpendicular to the fore axis has one of (i) a curvature in a plane perpendicular to the bore axis, and (ii) a closed perimeter.

Embodiments also provide a set of devices configured to retain a dispenser of liquid, the set comprising: an active device and a passive device. Each of these active and passive devices includes: (i) a base with a top side and a bottom side, the base having first and second base ends; (ii) a holder body having first and second sides and an opening formed therethrough along a first axis from the first side to the second side, the opening being sized to retain the dispenser having a nozzle; and (iii) an arm elongated along a second axis that is inclined with respect to the first axis, the arm having a first end affixed to the holder body and a second end affixed to the base. Each of the active and passive devices is dimensioned such that (when the dispenser is retained in the opening with the nozzle directed along the first axis and away from the holder body) a target region for delivery of a drop of the liquid contained in the dispenser is defined in a first plane that is parallel to a plane passing through the first and second base ends, the target region being substantially centered at the first axis. In contradistinction with the passive device from the set, the active device additionally includes, at its arm, a data-recording unit containing an optical system with a field-of-view (FOV) defined to cover and include (a) a tip of the nozzle, when the dispenser is retained at the opening, and (b) the target region. Alternatively or in addition, and for every device in the set: the base may be arcuately shaped and dimensioned to fit above and in contact with a user's nose bridge; the device may be dimensioned such that, when the base is on a user's nose bridge, the first passes through an eye of the user when; and the opening may have a cross-section in a plane transverse to the first axis, the cross-section being one of (i) a rectangular cross-section and (ii) as a cross-section in which the opening defines a curve having a constant radius of curvature. Alternatively or in addition, at least one of a length of the arm and an inclination of the arm with respect to the base, in a device from the set, is adjustable to position the first axis to pass through a user's eye once the base is disposed on a user's nose bridge. In a particular embodiment, at least one device in the set includes a wing portion affixed to the arm at a point between the holding body and the base and extending from the arm along an axis that is transverse to a reference plane, the reference plane containing the first and second axes. In one embodiment, and for each device in the set, the base is curved in a plane containing the first axis and a cross-section of the opening defined in a plane perpendicular to the first axis has one of (i) a curvature in a plane perpendicular to the bore axis, and (ii) a closed perimeter. In a specific implementation of the set, a structural configuration of the active device and a structural configuration of the passive delivery device are substantially the same with an exception of the data-recording unit present at the arm of the active device. In a related embodiment, the active device may include one or more of (i) a programmable computer-readable processor in operable cooperation with tangible non-transient storage medium, the processor configured to acquire optical data that have been collected by the data-recording unit and that represent a scene at the target region; and (ii) a sensor system configured to wirelessly communicate with a programmable electronic circuitry to produce a record of time schedule of actual use of the active device, that has been equipped with the dispenser, for drop delivery into the target region. In one implementation, an optical axis of the optical system intersects the first axis at the target region and, optionally, it intersects the first axis at an acute angle (the acute angle being an internal angle of a triangle defined by the first axis, second axis, and optical axes). The holding body may be shaped as a cuboid. Alternatively or in addition, for each device in a set the bottom side is curved with a center of curvature located in a plane containing both the first and second axes, the center of curvature being separated from the first end by a distance exceeding a length of the arm.

A related embodiment provides a method for using a set of devices configured to retain a dispenser of liquid. The set of devices includes an active device and a passive device, wherein each of the active and passive devices contains (a) a base having a top side, a bottom side, and first and second base ends; (b) an arm extending from the top side of the base at a first end of the arm; (c) a holder portion with an opening therethrough, the opening having an opening axis and sized to retain a liquid drop container having a nozzle, (here, the holder portion is affixed to a second end of the arm such that when a corresponding device is in operational position with the liquid drop container retained at the opening, a target region for delivery of drops emitted from the nozzle is defined in a first plane that is parallel to a plane passing through the first and second base ends, the target region being substantially centered at the opening axis). In comparison with and in contradistinction to the passive device in a set, the active device additionally includes, at its arm, a data-recording unit containing 1) an optical system with a field-of-view (FOV) defined to cover and include (i) a tip of the nozzle of the container retained in the bore, (ii) the target region, and (iii) a space separating the tip from the target region, and 2) an optical detector. Here, the data-recording unit is configured to record images of a scene within the FOV. The method includes the steps of: applying hand input to the active device to emit a liquid drop from the container retained by the active device in a direction of the target region while recording a series of image frames, each frame representing a corresponding position of the drop in the space; monitoring, with the use of the data-recording unit, whether the drop landed in, partially in, or out of the target region; and using the passive device to deliver a liquid drop from the container retained therein to the target region. In one implementation, the use is made of a device (in the set) for which a third plane tangential to a point at the top side and the second plane are parallel to one another. The step of using may include i) applying hand input to the passive device to emit a liquid drop from the liquid drop container retained in by the passive device in the direction and/or ii) starting to use the passive device to deliver the liquid drop from the container retained therein when results of the monitoring indicate successful and repeatable delivery of the drop to the target region.

In a specific embodiment of the method, at least one of the following conditions is satisfied: (i) one or more of the applying, and using includes self-administering of the drop by the user without supervision; (ii) the monitoring includes wirelessly monitoring via a remote unit; and (iii) any of the applying and using includes compressing the liquid container, that has been retained at the opening, against a lateral protrusion located between the holder portion and the base with one of (a) a finger, and (b) a level portion of the device hingedly attached to the holder portion while the base is positioned on a nose bridge of a user with the bottom side in contact with the nose bridge, and wherein the target region is the user's eye. In one implementation, each of the applying and monitoring is carried out after the using. The method may additionally include one or more of the following steps: (i) adjusting one or more of a position of the active device, an orientation of the active device, and the hand input based on results of the monitoring; and (ii) inserting the liquid drop container in the opening of the holder portion to retain the container along the opening axis with the nozzle pointing towards the target region. The step of inserting may include one of the following: (i) retaining a flat tail portion of a single-use squeezable liquid drop container in said opening, the opening defining a hollow through the holder portion, the opening axis being completely surrounded by the holder portion in a cross-section that is transverse to the opening axis; and (ii) retaining a cylindrical neck of a multiple-use squeezable liquid drop container in said opening, wherein the opening is formed between first and second prongs of the holder portion, the first and second prongs extending transversely to the arm. In a specific implementation of the method, at least one of the top side and the art includes indicia of location, and any of the retaining the flat tail portion and retaining the cylindrical neck includes positioning a tip of the nozzle substantially at a level of said indicia of location. Alternatively or in addition, the embodiment of the method may comprise one or more of: (i) positioning any of the active and passive device on a nose bridge of a user while bringing a cross-stabilizer portion of the device in contact with a forehead of the user, wherein the cross-stabilizer portion extends transversely from the art between the holder portion and the base; and (ii) with a programmable electronic circuitry, generating a user-perceivable report containing data that represent whether the drop landed in, partially in, or out of the target region, and further complemented with determining if a change of employing the active device to employing the passive device is appropriate (based at least in part on a figure of merit calculated from said data and representing success of a drop delivery to the target region the report).

### BRIEF DESCRIPTION OF THE DRAWINGS

The invention will be more fully understood by referring to the following Detailed Description of Specific Embodiments in conjunction with the not-to scale Drawings, of which:

FIGS. 1A, 1B illustrate, in perspective and top views respectively, a passive device structured to accommodate a single-use container of liquid according to an embodiment of the invention;

FIGS. 2A, 2B show the passive device of FIGS. 1A, 1B employed to retain a single-use liquid drop dispenser in a dispenser-accommodating opening of the device;

FIGS. 3A provide perspective views of a passive device according to a related embodiment of the invention; FIG. 3A: full device; FIG. 3B: a pitch-fork (bottle-holding) portion of the device;

FIGS. 4A, 4B, 4C illustrate a mechanism of retaining a multi-use liquid drop dispenser (or bottle, or container) in the device of FIG. 3 FIG. 4A: a process of insertion of the drop dispenser between the prongs of a pitchfork portion of the device; FIG. 4B: two multi-use liquid drop dispensers of different dimensions, one being kept/retained by the device of FIG. 3; FIG. 4C is a schematic diagram illustrating an example of the passive device of FIG. 3 dimensioned to accommodate and retain one of the dispensers of FIG. 4B;

FIG. 5 illustrates the use of the passive device of FIG. 3 for dispensing of liquid drops from the container retained in the device;

FIGS. 6A, 6B are perspective and side views, respectively, of a passive liquid-drop-dispenser holding device configured according to another embodiment of the invention;

FIGS. 7A, 7B are perspective and side views of an active dispenser-holding device structured according to an embodiment of the invention;

FIGS. 8A, 8B illustrate perspective and front views of an active device configured according to another embodiment of the invention;

FIG. 8C illustrates the device of FIG. 8A with a fluid container retained between the prongs of the pitchfork of the device;

FIGS. 9A, and 9B illustrate, in perspective and top views, a body of a specific embodiment of a passive device structured to accommodate a single-use container of liquid;

FIGS. 9C and 9D provide perspective and bottom views of a lever-portion of the specific embodiment that mechanically cooperates with the body shown in FIGS. 9A, 9B;

FIG. 10 illustrates the specific embodiment formed by mechanical cooperation of the body of FIGS. 9A, 9B and the lever of FIGS. 9C, 9D, and with a single-use container retained in the slit of the embodiment;

FIGS. 11A, 11B, and 11C provide perspective, bottom, and side views, respectively, of a passive device structured to accommodate a single-use container of liquid according to an embodiment of the invention;

FIG. 11D illustrates the embodiment of FIG. 11A with a single-use fluid container retained in the bore of the embodiment and pressed against the tongue protrusions, located on the extension of the body of the device;

FIGS. 12A, 12B, and 12C illustrates a passive device structured to accommodate a single-use container of liquid according to an embodiment of the invention. FIG. 12A: perspective view; FIG. 12B: top view; FIG. 12C side view;

FIGS. 13A, 13B are perspective and top views of an embodiment of a passive device structured to accommodate a multi-use container of liquid between the prongs of a pitchfork portion of the device which also features cross stabilizers;

FIG. 14 is an illustration of a plurality of single-use containers, accommodation of which is provided by a plurality of embodiments of the invention;

FIG. 15 is an active device structured to accommodate multi-use containers of liquid according to an embodiment of the invention;

FIGS. 16A, 16B, 16C, and 16D provide side and top and perspective views of a passive device structured to accommodate a single-use container of liquid according to an embodiment of the invention, and the associated steps of inserting a single-use container of liquid into the device;

FIG. 17 includes 4 (four) frames and illustrates an EDAM device (A), highlight of the proper eye drop application (B), an eye drop tip contacting eyelid and cornea (C), and multiple missed drop application (D);

FIG. 18 contains a table summarizing demographics of empirical study subjects;

FIGS. 19A, 19B are plots providing empirical comparison between the results of actual drop delivery to an eye vs. prescribed regimen. The overall percent actual treatment is not significant when compared with the perceived with actual (FIG. 19A). The percent absolute variation from the prescribed regimen (FIG. 19B) yields significant difference of perceived vs actual (p<0.001). Error bars represent standard deviation; NS implies (not significant);

FIGS. 20A, 20B are plots providing results of an “intention to treat”: a comparison between intention to deliver drops to an eye and the prescribed regimen. The overall percent intention to treat is not significant when comparing the perceived to actual (FIG. 20A). The percent absolute variation from the prescribed regimen between the two groups is highly significant (P<0.001). Error bars represent standard deviation; NS implies (not significant);

FIGS. 21A, 21B are plots providing information about the overall percent success rate (drops in/drops dispensed) and the percent absolute variation from the prescribed regimen of the study population is significant (p<0.007) when comparing the perceived with the actual. Error bars represent standard deviation;

FIG. 22 includes a table summarizing missed applications of droplets to an eye and occurrences of the contamination of a drop dispenser;

FIGS. 23A, 23B, 23C, and 23D illustrate an alternative embodiment of the invention;

Relative scales of elements in Drawings may be set to be different from actual ones to appropriately facilitate simplicity, clarity, and understanding of the Drawings. For the same reason, not all elements present in one Drawing may necessarily be shown in another.

### DETAILED DESCRIPTION

In accordance with preferred embodiments of the present invention, methods and apparatus are disclosed for solving the operational shortcomings of current devices and methodologies employed to aid in the delivery of a liquid drop to a region of interest (ROI). In particular, a shortcoming of current liquid drop-dispensing devices is their inability to conform to the specific needs of a particular user. This shortcoming is addressed by providing an operationally-adjustable device and a plurality of such devices, as a kit, that can be fit to a user's unique needs based on the user's anatomy (e.g., high nose bridge versus a low nose bridge) and/or any other physical traits of the user (e.g., tremors, arthritis, etc.), as well as the type of the used liquid container (e.g., single-use, multi-use, length, etc.).

Another shortcoming of current devices and methods employed to aid in the delivery of a liquid drop to a region of interest is the lack of means to evaluate and teach/train the user the correct way to effectively deliver a liquid drop to the ROI, specifically when the user is primarily attempting to deliver the drops in his/her at-home environment and without supervision. (Such situation is explained in detail in the accompanying Appendix, which provides the results of several clinical studies investigating this problem.) This shortcoming is addressed by providing two or more liquid drop delivery devices (configured to hold and retain liquid dispensers or drop dispensers such as containers of fluid), where at least one device is “active” (in that it has an event registration/feedback system such as an optical system or a sensor system, for example, configured to collect data and provide feedback about whether a drop is successfully delivered to the ROI or indicating whether and when the device is being used), and at least one other device is “passive” (in that, optionally being otherwise substantially structurally similar in configuration and operation to the “active” device, it does not have such a feedback system and is configured to simply hold, stabilize, and/or position a liquid-drop container to aid in successful delivery of the liquid drop(s).

An additional structural feature employed in an active device may be a feedback-generation capable system that is configured to monitor the process of delivery of the liquid drop, and/or to collect data regarding the user's delivery, or attempted delivery, of the drop to the ROI. The method for use of the array of devices includes operational transitioning between active and passive devices depending on the user's needs. If the user has not mastered the intended delivery technique, an active device can be used to monitor and track the drop delivery data. Depending on the feedback data, adjustments can be made to the user's delivery technique. For example, once the actively collected data indicate that drop delivery is successfully occurring as intended (either spatially, with respect to the targeted ROI, or temporally, in terms of the delivery on schedule, or both), the user can transition to using a passive device. Accordingly, the process of using the platform or set or kit of devices includes a user's transitioning from employing the active device to employing the passive device (once the data collected by the feedback system indicate that the user's drop-delivery-to-the-ROI technique is successful). In a related example, if the user has other specific needs (for example, they user forgets to deliver the liquid at the appropriate times), an active device used at the time can be additionally equipped with a sensor system that can be paired with a smartphone or other wireless application, which is programmed to remind the user to use the liquid drops if and/or when the device is not used at the appropriate times. Alternatively, the process of using the set may include the user's transitioning from employing the passive device to employing the active device (once the collection of feedback data is prompted by lack of success of using the “passive” device on the user's part, to begin with).

Yet another shortcoming of current devices is the problem caused by the inability of existing liquid-drop-delivery aids and monitoring systems to accommodate single-use containers and/or dispensers of liquid (to which related industry is transitioning) This deficiency is solved by providing an embodiment of a device configured to contain a cavity, judiciously sized to retain a single-use liquid drop dispenser, while the device also has an optional built-in feedback system (such as an optical means for continued monitoring of the liquid drop delivery to the ROI or a sensor system for indicating whether and when the device is being used).

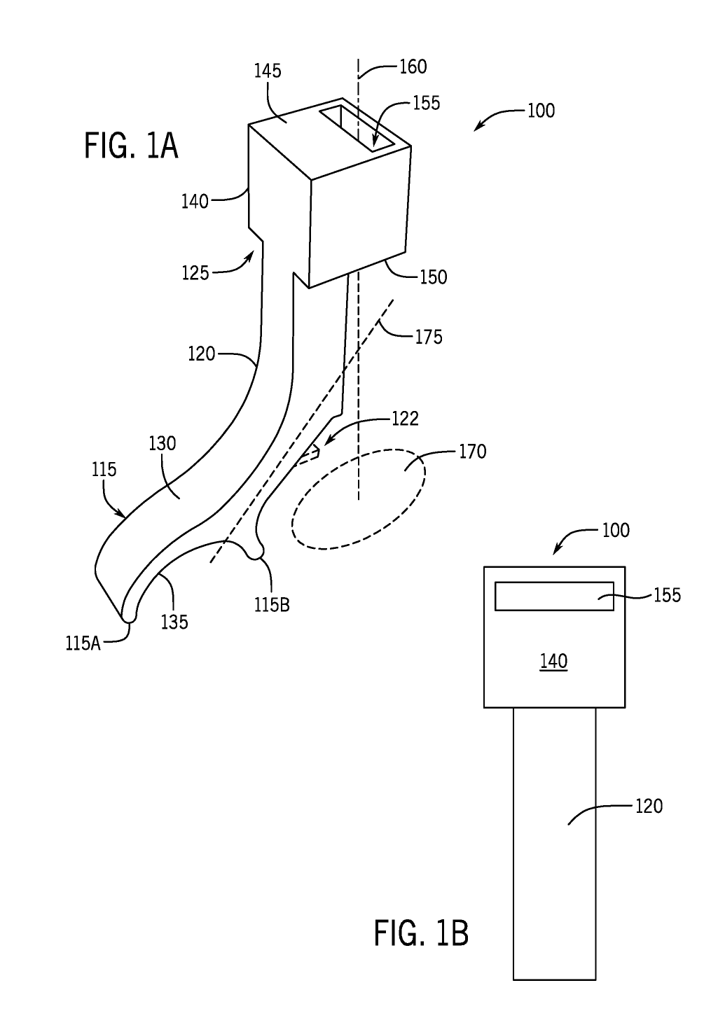

While various Figures discussed below show specific embodiments of either a passive device or an active device, it is appreciated that all embodiments of the invention generally have in common several structural components and/or characteristics, regardless of a particular configuration, orientation, and/or dimensions of such components. Preferably, all embodiment are made from material(s) lending themselves to injection molding, lathe machining, or 3D printing. Such common components are now introduced in reference to FIGS. 1A, 2A, 4C A base or foot 115; an arm 120 extending from the base 115; and a holder (or holder portion) 125 typically connected to the opposite end of the arm 120 are what may be referred to as the main of such components. The base 115 typically has a top side 130 and bottom side 135. The bottom side 135 of the version of the base 115 shown in FIG. 1A is shaped to deviate from being linear—generally, curvilinearly or to form an angle, arcuately as shown—to define two ends of the base: the end 115A and the end 115B. Generally, the curvilinear shape of the base 115 can be part of a round, elliptical, or parabolic curve, to name just a few.

The arm 120 extends from the top side 130 of the base 115 and connects at its other end to the holder 125. The holder 125 includes a body 140, which has a first surface 145 and a second surface 150 (as shown—upper and lower surfaces of the body 140), and a bore or cavity or hole or recess or space 155 that is formed through the body 140 and extends from the first surface 145 to the second surface 150 along a bore/cavity/hole/recess/space axis 160.

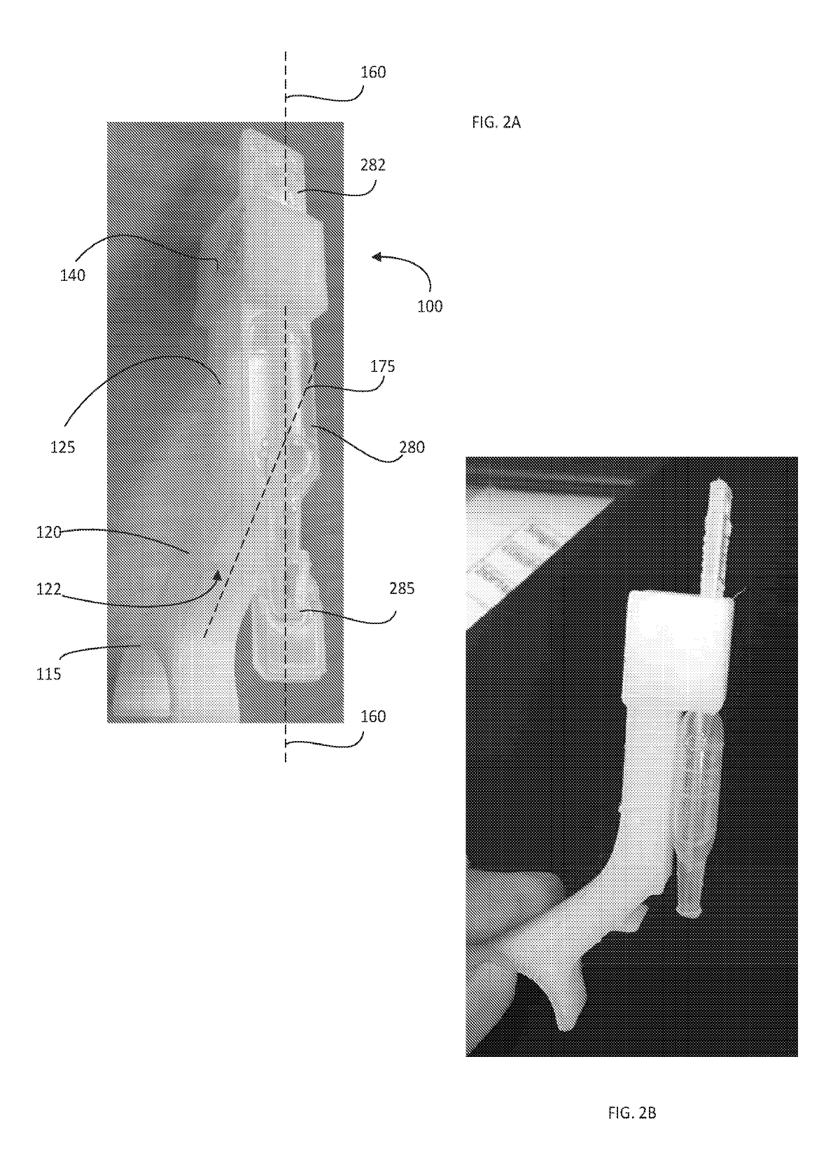

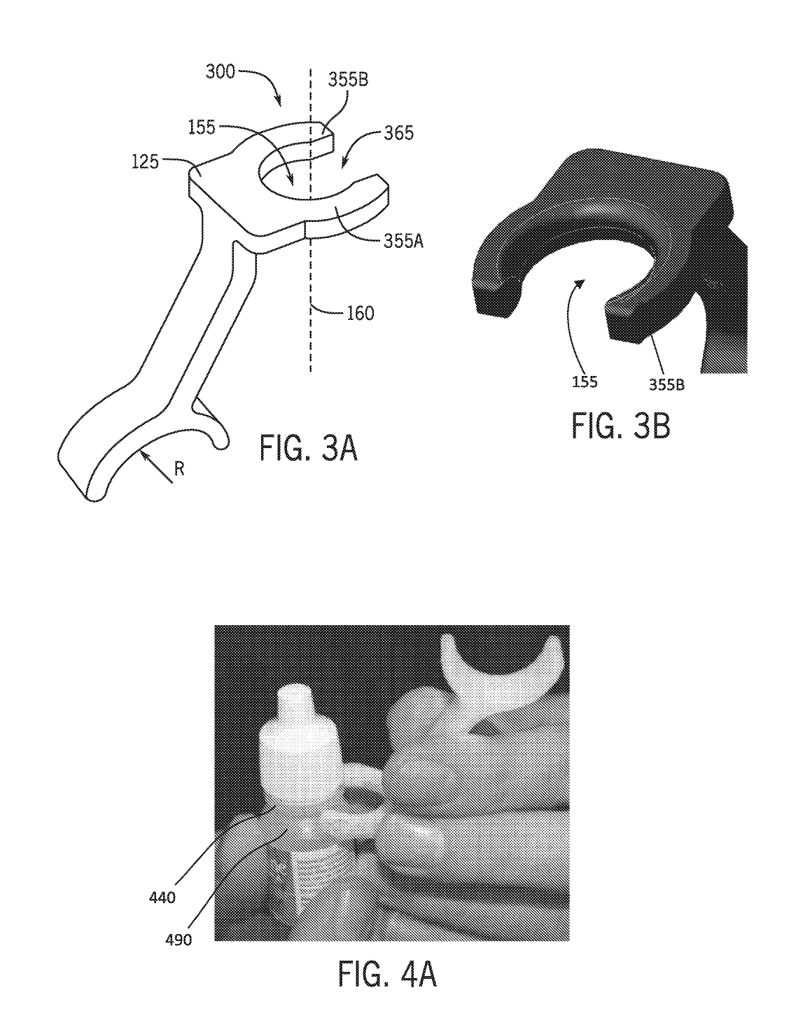

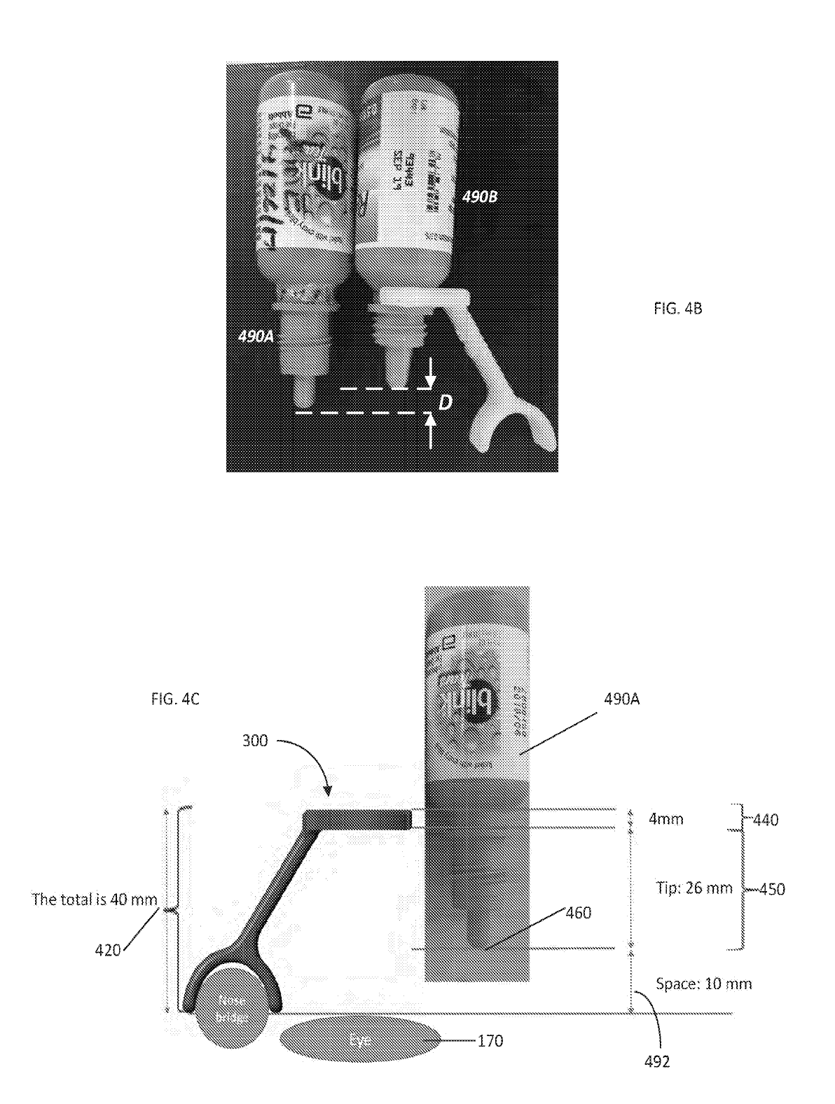

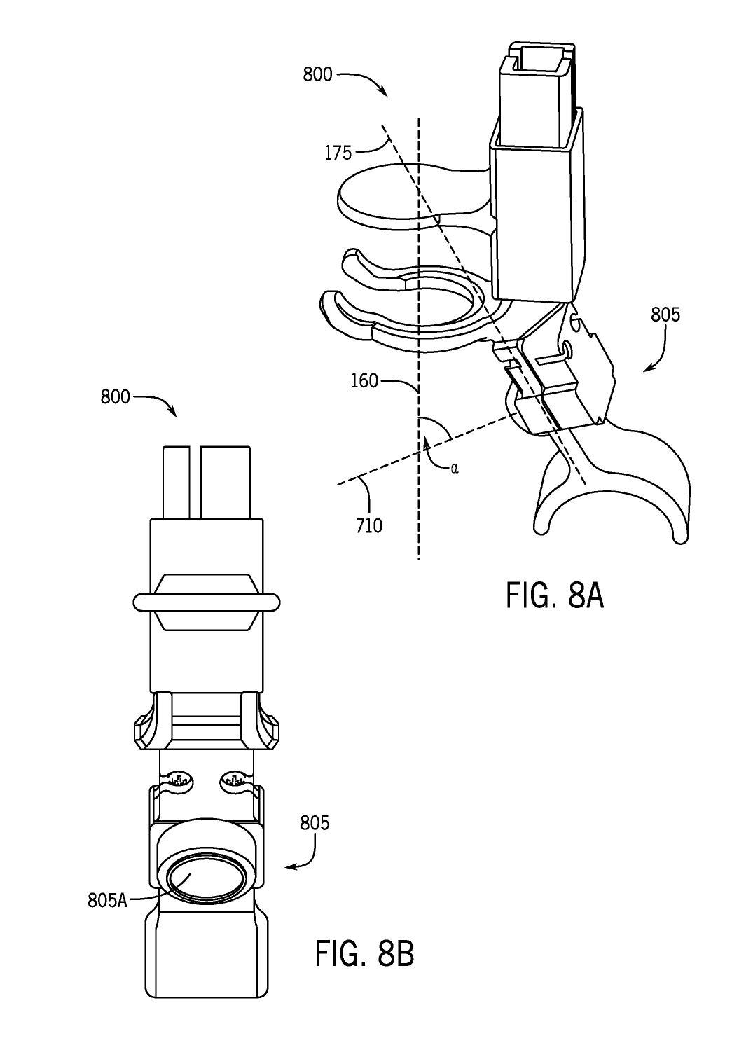

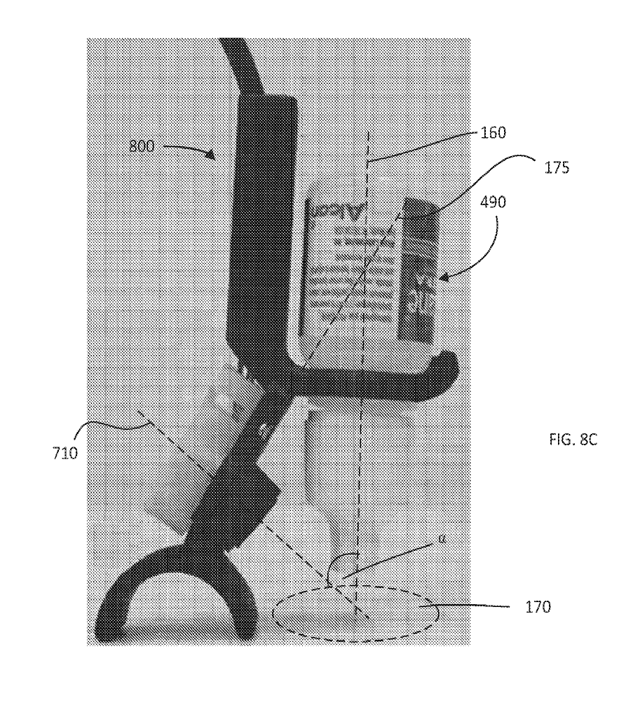

Generally, the bore 155 can be fully closed on four sides that circumscribe/surround the first axis 160, as shown in embodiment 100 of FIGS. 1A and 2A. Alternatively, the bore 155 may not be completely closed on one of the four sides and may have a slit or opening along a side of the body 140 for example, along the third side 365 of the body 140 (as is shown later in embodiments 300, 800 of FIGS. 3, 8A, in which cases the holding body with a bore there through is shaped as a pitchfork). Alternatively, the bore 155 may also not extend completely through the body 140 and may be dimensioned as a cavity sized to retain only an end or a tail of a container of liquid. Stated differently, depending on a particular implementation of the device, the bore or cavity 155 can be configured as a hollow throughout the body 140 (such that the cross-section of the hollow is circumscribed by the material of the body 140) or as a cavity with an opening defined in a direction that is substantially perpendicular to the bore axis 160. Also, and depending on a particular implementation, the bore 155 is dimensioned to support a corresponding liquid-holding container either at the peripheral or tail portion of the container (shown schematically in FIG. 2A as an end of the container opposite to the container's nozzle 285) or at the container's intermediate portion (such as a neck 440) located between the nozzle and the opposite end of the container (as shown, for example, in FIG. 4C). Accordingly, the bore is characterized by a cross section (defined in a plane transverse to the bore axis 160) that is substantially rectangular or that defines a curve with a constant radius of curvature (that is, the radius of curvature of a wall of the bore is the same at any point of the wall of the bore). Notably, in embodiments in which the foot or base portion is curved and in which the holding portion defines a pitchfork, based portions and the holding portion are oriented in planes that are transverse to one another. Specifically, the pitchfork-like shaped portion lies in a plane that is perpendicular to the bore axis 160, while the curvature of the foot portion is defined in a plane that contains the bore axis 160 (see, for example, radius of curvature R in FIG. 3).

When the devices are in their operational position, the holder 125 is oriented above the base 115 and the region of interest (ROI) 170 is located substantially below the bore 155 (and, optionally, in a plane that is parallel to the plane passing through the ends 115A, 115B).

The arm 120 is affixed to the body 140 at the arm's first end and extends from the body 140 generally along a second axis 175 that is inclined with respect to the first axis 160. The arm 120 has its other end affixed to the base 115. When the base 115 has an arcuate shape, the center of curvature of the arc may be chosen to lie in a plane that contains both the first axis 160 and the second axis 175, and such that the center of curvature is separated from the first end of the arm 120 by a distance exceeding a length of the arm 120. In one implementation, where the base 115 is chosen to be shaped as substantially half-an-ellipse (with 16 mm minor axis and 20 mm major axis, or, alternatively, a half round with a 20 mm diameter; with the thickness of the body of the base 115 is about 2.8 mm, while the width is about 10 mm) the distance between the axis 160 and the center of the base (nose bridge) 115 is 31 mm, resulting in that the center of region of interest 170 is at a separation distance of about 31 mm away from the center of the nose bridge during the operation of the device, to address a typical distance between eye-pupils of a typical person of about 58 mm to about 66 mm. This separation distance can be customized for patients.

These general structural features are judiciously chosen to ensure that both the active and the passive versions of a particular embodiment are substantially structurally similar to one another, and can be used interchangeably by the same user with minimal—if any—deviation from the established drop-delivery procedure to which the user became accustomed while using one of the versions of such device.

### Passive Embodiments and Methods of Operation of Same

Example 1: Drop-Dispenser for Single-Use Container. Referring now to FIGS. 1A, 1B and 2A, 2B, an embodiment 100 of a passive device structured to accommodate single-use containers of liquid that are becoming prevalent in related industry is shown to have a bore 155 with a polygonal (for example, rectangular) cross-section in a first plane that is transverse to the first axis (the axis of the bore) 160. Here, the cross-section of the bore in a plane perpendicular to the axis 160 has a closed perimeter. The bore 155 as shown is defined by bore walls that are substantially parallel to the first axis 160 and limited by the upper and lower surfaces 145, 150. The bore 155 is appropriately dimensioned to tightly hold a single-use container of liquid 280, a peripheral substantially flat end portion or tail 282 of which is reversibly inserted or placed into the bore 155 such that the body and the nozzle 285 of the container are extending alongside the arm 120, as shown in FIG. 2. When the single-use container 280 is properly affixed in the bore 155, its nozzle 285 is pointing generally along the arm portion of the device and generally towards the base and/or the area neighboring the ROI. In a specific case, the first axis 160 points towards the ROI 170. The ROI is generally defined in a plane parallel to the plane that contain both ends 115A, 115B of the base 115 and substantially centered at the center axis 160 of the bore 155. The single-use container 280 is generally made of pliable material such that it can be effectively manipulated to emit a single drop when squeezed. In operation, once secured in the bore 155, the single-use container 280 is situated next to the arm or arm portion 120 such that the container 280 may be compressed against a side of the arm or arm portion 120.

Additionally, the single use container device may contain a marking or reference element 122 (such as a protrusion or indentation or another indicia of location on a surface of the arm 120) configured to indicate a level or a point with respect to which (for example, above which) the tip of nozzle 285 of the single-use container 280, after opening, should be placed for use in a particular application (In a non-limiting and a very specific example, in an application of a drop to the eye, such marking element serves to reduce risk of contact of the container 280 with the eye).

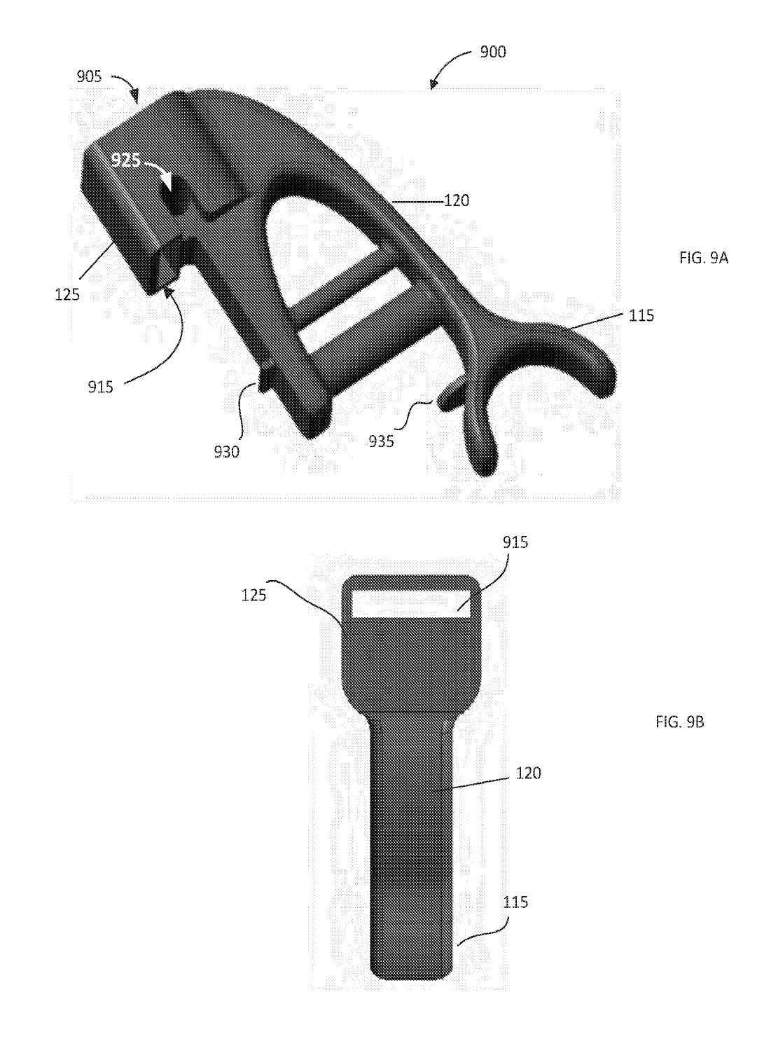

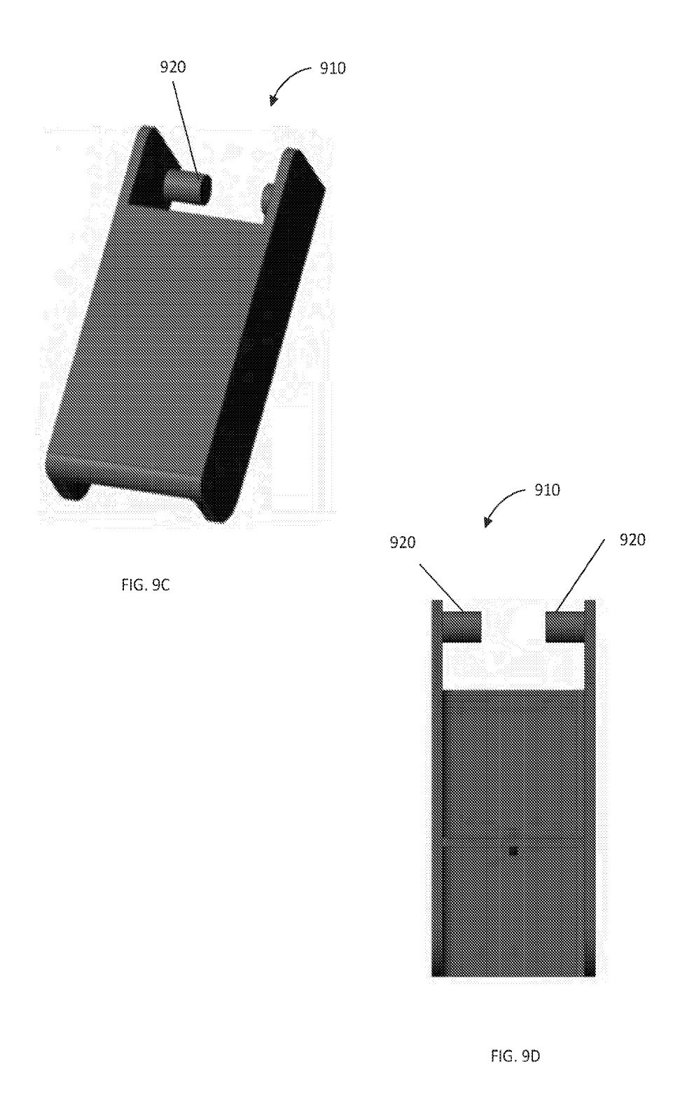

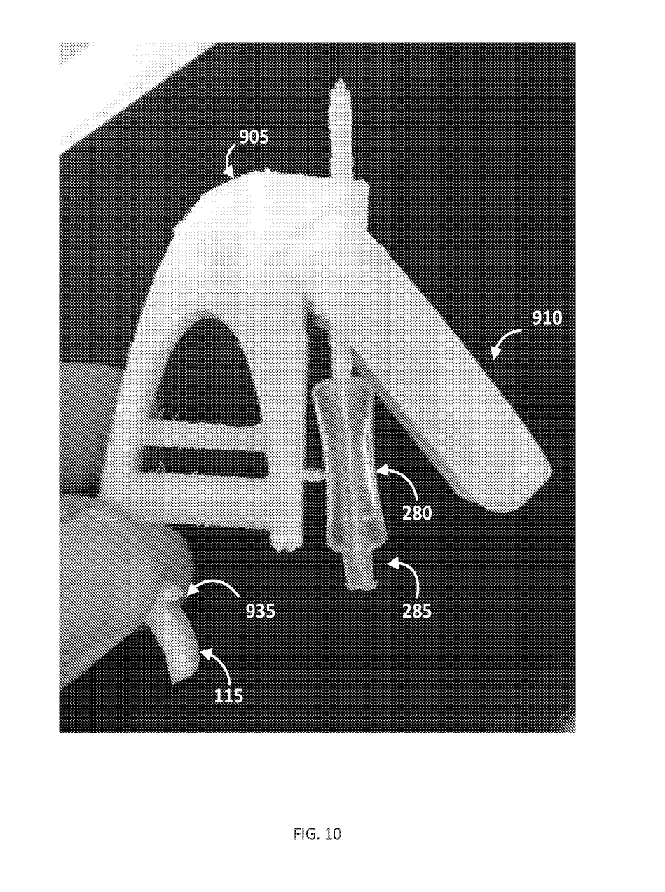

Example 2: Drop-Dispenser for Single-Use Container. According to the idea of the invention, various embodiments of passive devices are tailored to specific needs of a user of the device(s). For example, the embodiment 900 shown in FIGS. 9A, 9B, 9C, and 9D includes the main portion or body 905 with already-described above, main components (such as base 115, arm 120, and holder portion 125), but in addition incorporating a separate, removably integrated with the main portion 905 of the embodiment component 910 (referred to, for simplicity, as cover or lever) to facilitate the squeezing of the single-use container 280 once the container is installed and retained in the bore 915 of the holder 125 (as shown in FIG. 10). A lever 910, which is shown as a stand-alone, disconnected from the body 905 element in FIGS. 9C, 9D, is configured to be attached to the body 905 of the embodiment 900 via co-axial protrusions 920 (that extend towards one another from the facing-each-other surfaces of the lever 910) and that are, when assembled with the body 905, accepted into receptacles/apertures 925 formed on opposite sides of the holder portion 140 of the body 905. When assembled with the body 905 as shown in FIG. 10, the lever 910 is connected to the body in such a manner as to make the protrusions 920 and receptacles 925 substantially co-axial with each other and to allow the lever 920 to freely pivot about the axis that is common to the protrusions 910 and the receptacles 925 and that extends through the centers of the receptacles 925. This lever 910, when assembled with the body 905 to form the embodiment 900, makes it easier for a user to compress an inserted/retained single-use container 280 by providing a greater surface area with which to apply pressure. When the user applies pressure to the lever 910 in a direction towards the retained container 280, the single-use container 280 is pressed against protrusion or tongue 930, which further aides in helping the user to effectively emit the desired number of drops from the container 280.

The foot or base 115 is shown equipped with a shelf or protrusion 935 (which, in the alternative implementation can also be formatted as an indentation or another indicia on a surface of the foot or base 115), and configured to indicate a level or a point with respect to which (for example, above which) the tip of nozzle 285 of the single-use container 280, after opening, should be placed for use in a particular application (in a non-limiting and a very specific example, in an application of a drop to the eye, such marking element serves to reduce risk of contact of the container 280 with the eye). In other words, the element 935 is configured to assist with proper positioning of the drop bottle tip when such bottle is retained in the embodiment of the drop dispenser.

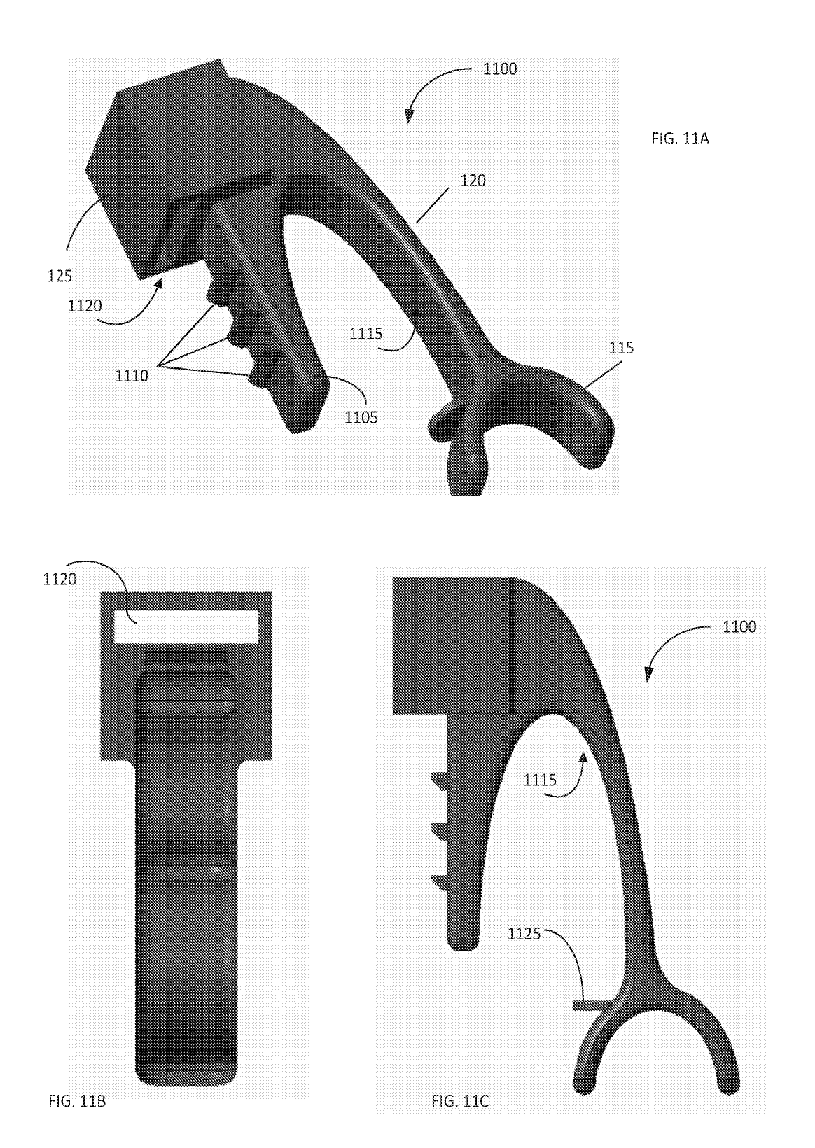

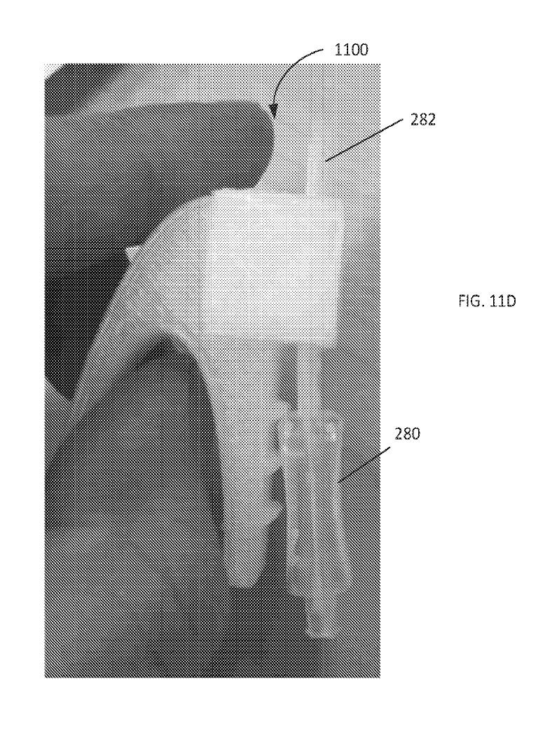

Example 3: Drop-Dispenser for Single-Use Container. The embodiment 1100 shown in FIGS. 11A, 11B, 11C is similar to that of the embodiment of FIG. 9A, 9B, 9C, however, it does not include and operates without the use of the lever (such as lever 910 of FIG. 10). In addition to the main components (such as base 115, arm 120, and holder 125), this embodiment may include an extension 1105 protruding downwardly from the holder portion 125 (and extending generally in the same direction in which the arm 120 extends from the holder portion 125), equipped with tongues 1110 on its front surface (the surface facing away from the arm 120). As shown, the extension 1105 and the arm 120 mechanically cooperate such as to define a continuous surface 1115, shown in FIGS. 11A, 11C as an arcuate surface. In the process of use of the embodiment 1100, the user's thumb may be positioned in the arcuate gap formed between the arm 120 and the extension 1115, as illustrated in FIG. 11D, thus providing the user with an effective holding position. FIG. 11D shows embodiment 1100 retaining a single-use container of liquid 280 at its peripheral portion 282.

The tongues 1110 aid in causing a single-use container 280 (once inserted and retained in the bore 1120) to emit the desired amount of fluid (for example, a pre-determined number of drops) when squeezed or pressed against the tongues 1110. The multiplicity of tongues 1110—as opposed to a single, only tongue—allows, in operation of the device with different single-use containers, for compression of differently-shaped single-use containers (the outer surface of some of which may be concave, as that in FIG. 11D, while the outer surface of others may be convex in shape). Similar to the embodiment shown in FIG. 10, the foot or base 115 may be equipped with a shelf or protrusion 1125 (which, in a related embodiment can also be configured as an indentation or another type of indicia on a surface of the foot or base 115), and configured to indicate a level, or a point with respect to which (for example, above which) the tip of nozzle 285 of the single-use container 280, after opening, should be placed for use in a particular application. The feature 1125, generally, is employed to properly position the tip of the liquid container (when retained in the device 1100, such that the tip is no lower than the level of the feature 1125.

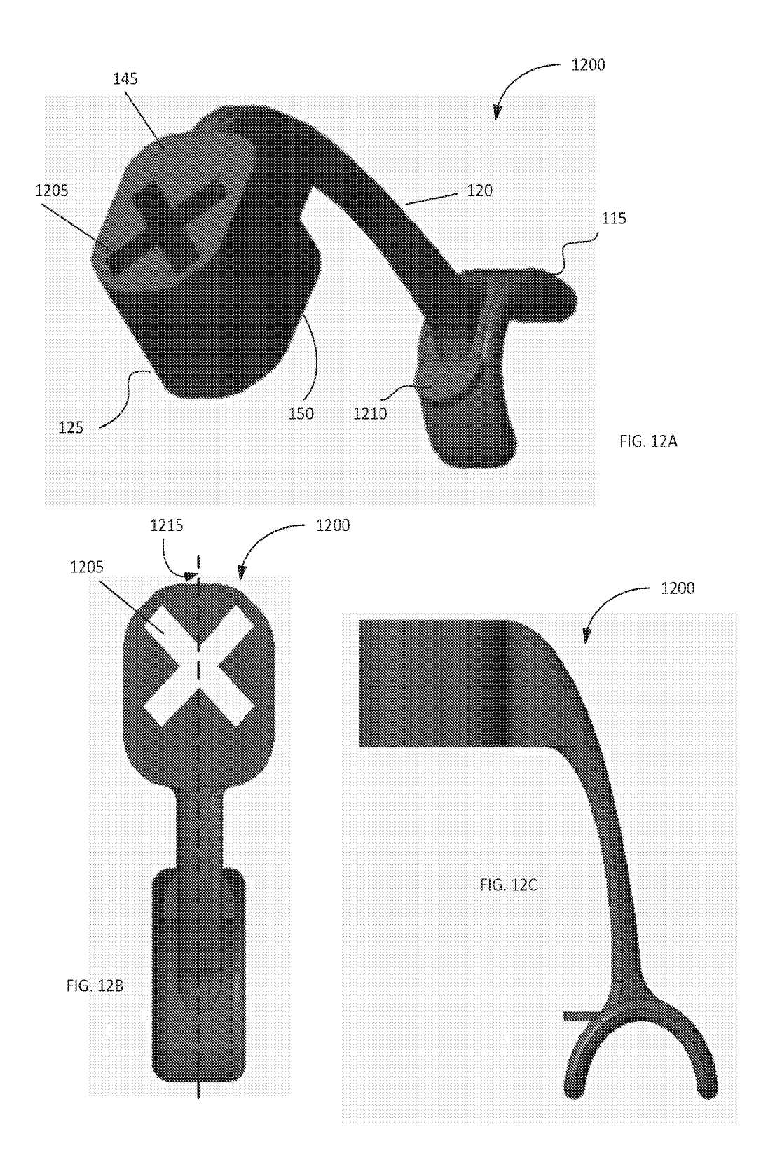

Example 4: Drop-Dispenser for Single-Use Container. The embodiment 1200, shown in FIGS. 12A, 12B, and 12C, also a passive device structured to accommodate single-use containers of liquid, includes the main components (base 115, arm 120, and holder 125) but with the a bore or slit 1205 having a cross-shaped cross-section in a first plane that is transverse to the axis of the bore 1205. The bore 1205 as shown is defined by bore walls that are substantially parallel to the axis of the bore 1205 and limited by the upper and lower surfaces 145, 150. The bore 1205 is appropriately dimensioned to tightly hold a single-use container of liquid 280 in two positions, where each position is angled at forty-five degrees with respect to plane 1215 defined by a central vertical cross-section of device 1200, as shown in FIG. 12B. This angled position of the single-use container 280 aids the user in effectively pinching and squeezing the container while also manipulating the device 1200. The two positions allow the user to switch the device 1200 in the opposite direction and accordingly switch the angle of the container. Similarly to the previously described embodiments, the foot or base 115 may be equipped with a shelf or protrusion 1210 which can also be an indentation or another indicia on a surface of the foot or base 115, and configured to indicate a level, or a point with respect to which (for example, above which) the tip of nozzle 285 of the single-use container 280, after opening, should be placed for use in a particular application.

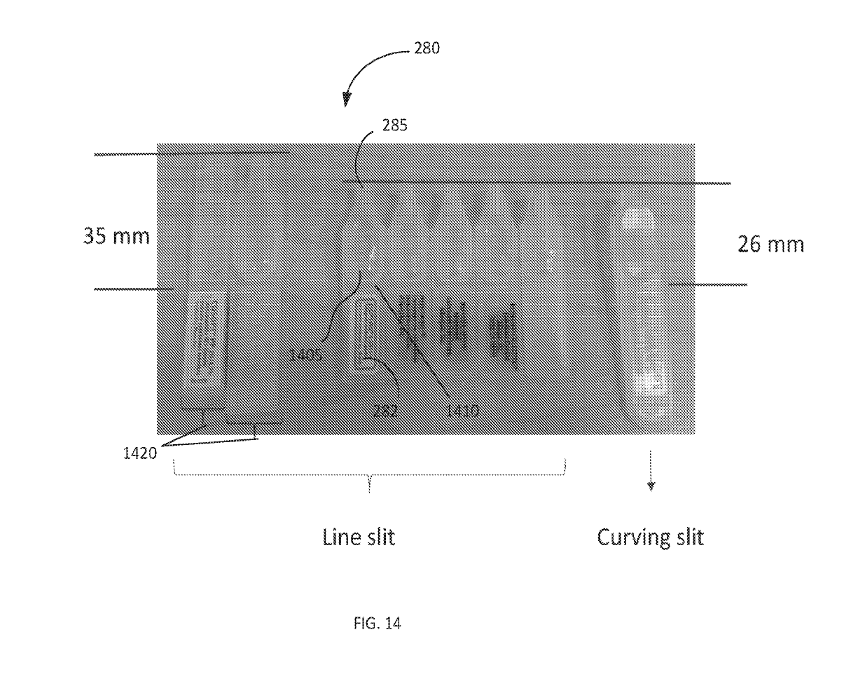

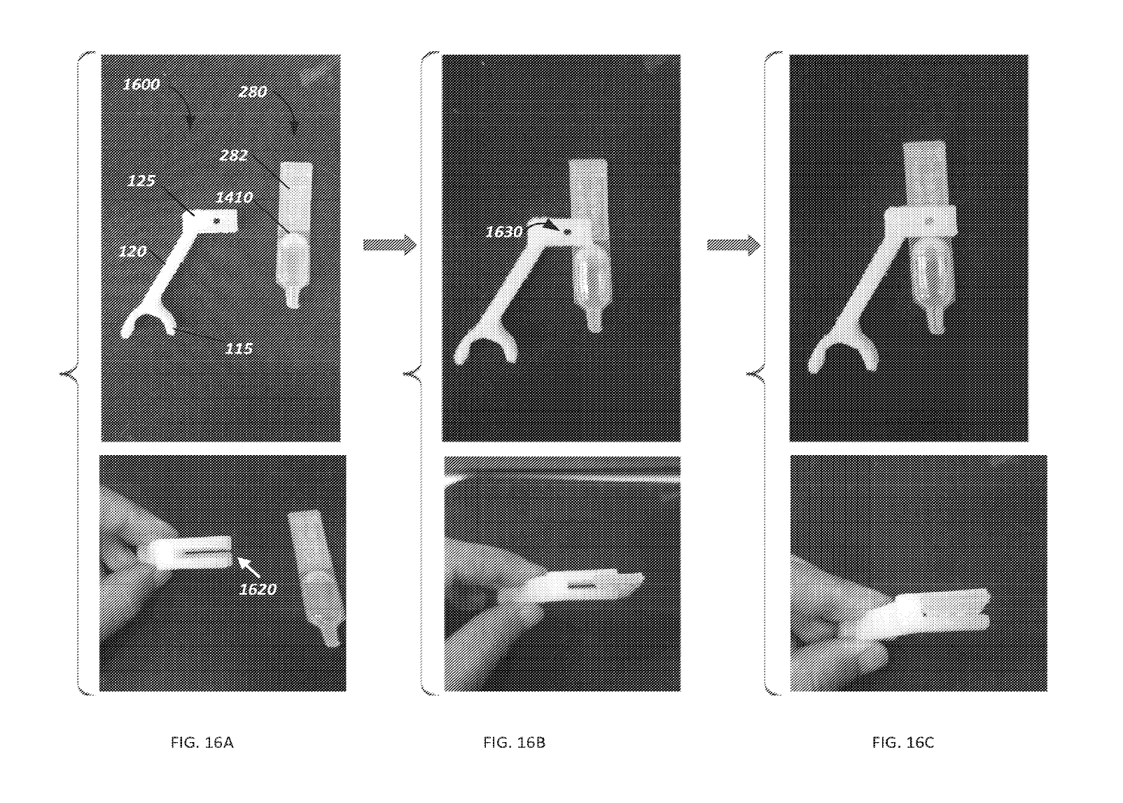

Example 5: Single-Use Containers of Fluid. Referring to FIG. 14, various examples of single-use containers of liquid 280, available commercially, are shown. Containers of different brands may have differing lengths (defined by a longitudinal extent of a given single-use container from the drop dispensing tip of the nozzle 285 to the opposite end of the liquid holding portion of the container 1405, and most commonly adhering to one of two accepted standards of 26 mm and 35 mm. In terms of shapes, the most prevalent designs include a trench or slit-like indentation in a body of the container (shown as t 1410) is formed between the liquid holding portion or volume 1405 and the peripheral or tail portion 282 of a given container. The previously described embodiments related to holding a single-use container of liquid 280 are designed to hold and retain a peripheral/tail portion 282. A related embodiment of the holder of a single-use container of fluid, shown in FIGS. 16A, 16B, 16C, and 16D is designed to hold the single-use container of liquid 280 at the slit 1410 (which, as shown in FIG. 14, may be linearly or curvilinearly shaped). In order to accommodate containers of varying lengths, the device illustrated in FIGS. 16A, 16B, 16C is provided in at least two incarnations each having a respectively-corresponding height, which is generally attained by modifying the length and angular orientation of the arm 120 with respect to the foot portion 115. Similarly, the embodiment described in FIGS. 16A, 16B, 16C is also provided in formats that can accommodate both a line slit and a curved slit type indentation of a single-use container. In particular, FIGS. 16A, 16B, 16C provide side and top views of a related embodiment 1600 of a passive device structured to accommodate a single-use container of liquid. Figs, 16A, 16B, 16C also illustrate associated steps for inserting a single-use container of liquid into the device 1600. Each of FIGS. 16A, 16B, 16C includes two sub-illustrations (shown embraced with a bracket in a corresponding Figure). The device 1600 includes the main components (base 115, arm 120, and holder 125); however the holder 125 defines an opening or bore 1620 on a side of the holder 125 to form the holder 125 in a shape of a tuning fork. Unlike the previous embodiments structured to accommodate a single-use container of liquid, the bore 1620 does not have walls that are substantially parallel to the axis of the bore, rather, there is a narrowing towards the bottom of the bore 1620 that correlates with the slit 1410 present on the various single-use containers of liquid. This narrowing of bore 1620 allows the tight positioning of slit 1410 of the single-use container of liquid 280 as shown in FIGS. 16B, 16C. FIG. 16B shows the single-use container of liquid 280 partially inserted into the device 1600, while FIG. 16C shows the single-use container of liquid 280 in the fully inserted position. Stated differently, a single-use container of liquid 280 can be slid into the holder 125 of the device 1600, and the narrowed portions of bore 1620 fitted snugly within the slit 1410, while the un-narrowed portions of bore 1620 securely hold the peripheral portion 282. A small hole 1630 is provided to allow the user to mark the middle position of the single-use container of liquid 280 because the width 1420 of the various single-use containers varies with the different brands.

Example 6: Drop Dispenser for Multi-Use Container. Referring to FIGS. 3A, 3B, 4A, 4B, 4C, and 5, a related embodiment 300 of a passive device structured to accommodate multi-use containers of liquid has a bore 155 with a substantially cylindrical cross-section in a first plane that is transverse to the first axis 160, where the body 140 further defines an opening at a third side 165 of the bore 155. Stated differently, the bore 155 in these embodiments defines the space between the prongs 355A, 355B of the pitchfork portion of the embodiment 300. Such semi-opened on the side bore (and/or pitchfork portion itself) is dimensioned to hold a multi-use container of liquid 490, the neck 440 of which is inserted or placed in the bore 155 through the opening on the side 365, as shown in FIGS. 3A, 3B, 4A, 4B, 4C and 5. Comparison between the implementations of the bore or cavity 155 shown in FIGS. 3A and 3B illustrates that the surface defining the cavity may contain angles and/or may be fully differentiable and smooth. (Generally, in embodiments the holder 125 of which is substantially similar to that of FIGS. 3A, 3B, For all devices having 355A and 355B, The thicknesses of the prongs 355A, 355B are about 4 mm. inner circle formed by the cavity 155 and embraced by the prongs 355A and 355B is about 14 mm in diameter; the opening/edge separation 365 between the ends of the prongs 355A and 355B is about 12 mm. These specific dimensions can be, of course, customized for specifically-shaped liquid containers.)

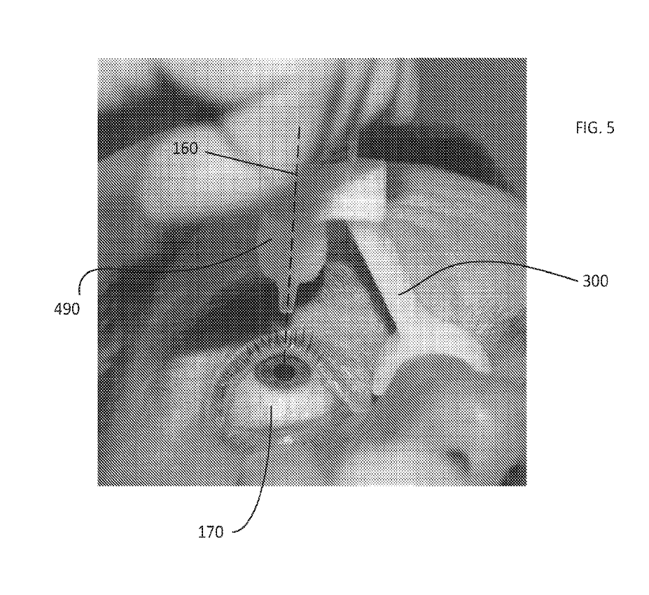

Example 7: Multi-Use Containers. Referring to FIGS. 4B and 4C, two examples of multi-use containers of liquid 490A, 490B are shown. For purposes of the device design, one measurement of conceal is the length of the tip 450 of the bottle 490 which is defined as the linear distance between the neck 440 of the bottle and the nozzle 460 which emits the drop, as shown in FIG. 4C. Generally, the length of the tip s50 of various multi-use containers differ, with most prevalent (adhered to in industry) lengths being either 26 mm or 20 mm. In reference to FIG. 5, when verifying empirically the practicality of an embodiment of the dispenser holder, it was determined that if the space or separation between the tip of the nozzle and ROI 170 is too small, the tip may be inadvertently brought in contact with the ROI 170 or surrounding areas (which could result in contamination of the tip or discomfort for the user). Alternatively, if the space is too great, the success rate of delivery of the drop to the ROI is decreased. Based on clinical testing, the optimal distance 492 between the tip of the nozzle of the bottle (container) retained in the holder and the ROI 170 was found to be about 10 mm. The optimal range, of course, can vary in different embodiments depending on a patient's application technique and other physical impediments such as arthritis, spinal fusion, a tremor or other physical difficulties. For example, if a patient is having difficulty successfully delivering to the desired ROI—a shorter space distance may be helpful. In a different example, if the tip of the eye drop bottle is contacting the eye lashes (when the device is in contact with the bridge of the nose), for example if the eye lashes are very long, a longer distance 492 might help to reduce the possibility that the tip of the nozzle will contact the lids and the resulting contamination and/or blinking the contact often causes. (Blinking at the wrong time can interfere with successful drop delivery to the ROI.) Variations in length for these reasons can occur of up to or around 6 mm shorter or longer. Accordingly, the dimensions of a passive device 300 have been configured to allow for retaining of differently sized fluid dispensers while, at the same time, providing for approximately optimal separation between the tip of the nozzle and the ROI. For example, in one embodiment a height 420 of 40 mm used in conjunction with a bottle with a tip length of 26 mm results in an approximate distance of 10 mm to the ROI 170, as illustrated in FIG. 4C. Similarly, an overall height of device 300 of about 34 mm provides the preferred optimal distance when used with a bottle with a 20 mm long tip. The exact distance between the tip of the nozzle 460 and the ROI 170 (for example, an eye) will vary slightly in practice, depending on specific and different facial features of a user of the system. For example, it may be preferred that systems/embodiments employed by a user having a low nose bridge (such as a user of Asian descent) are structured and/or dimensioned to provide for a tip-to-ROI separation that is slightly shorter than 10 mm, while a user having a higher nose bridge (such as a user with Northern European lineage) might be better served with a differently-dimensioned embodiment providing a tip-to-ROI space that is slightly larger than 10 mm. In other words, the variability of the facial features of different users should be taken into account when structuring a particular embodiment of the invention.

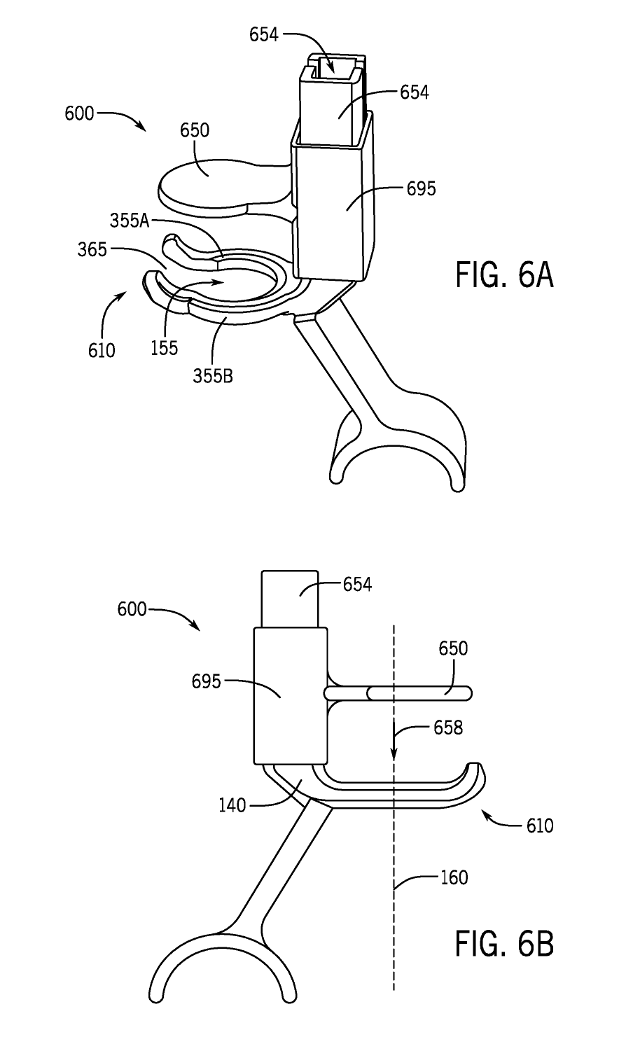

Example 8: Drop Dispenser for Multi-Use Container. Referring to FIGS. 6A and 6B, a related embodiment 600 of a passive device (structured to accommodate multi-use containers of liquid) has a bore or cavity 155 that defines the space between the prongs 355A, 355B of the pitchfork portion 610 of the embodiment 600. Similar to embodiment 300, such semi-opened on the side bore or cavity (and/or pitchfork portion itself) is dimensioned to hold a multi-use container of liquid 490, the neck 440 of which is inserted or placed in the bore 155 through the opening on the side 365. In a specific case, the embodiment 600 may have an optional bottle/container-securing mechanism 695. A portion of the mechanism 695 is a platform 650, a position of which is adjustable along the axis 160 to accommodate and retain multi-use containers of different sizes, as shown in FIG. 6. In one implementation, the optional securing mechanism 695 is spring-loaded (with respect to the upper portion 654 of the body 140) to have its position biased towards the location of the body 140. (The spring loading arrangement is not shown in FIG. 6, but it may be disposed in a hollow 654A of the portion 654, for example.) Once the bottle 490 is loaded between the platform 650 and the pitchfork. As a result of the spring-loading, the platform 650 forms a vectored-force 658 applied to the bottle 490 in the direction from the platform 650 towards the pitchfork 610 (which effectively squeezes and retains a multi-use bottle 490 in the correct position with its neck 440 cradled between the prongs 355A, 355B of the pitchfork portion 610). Additional features of this or any of the devices described may include a light or multiple light sources, such as LED or bulbs, to help users to position the device properly, particularly those users with poor vision. Such light(s) may aid in imaging of the application, and be used in combination with a recording system, which is more fully described below. The devices may also include mechanical stabilizers, to help those users with tremors, also described in greater detail below.

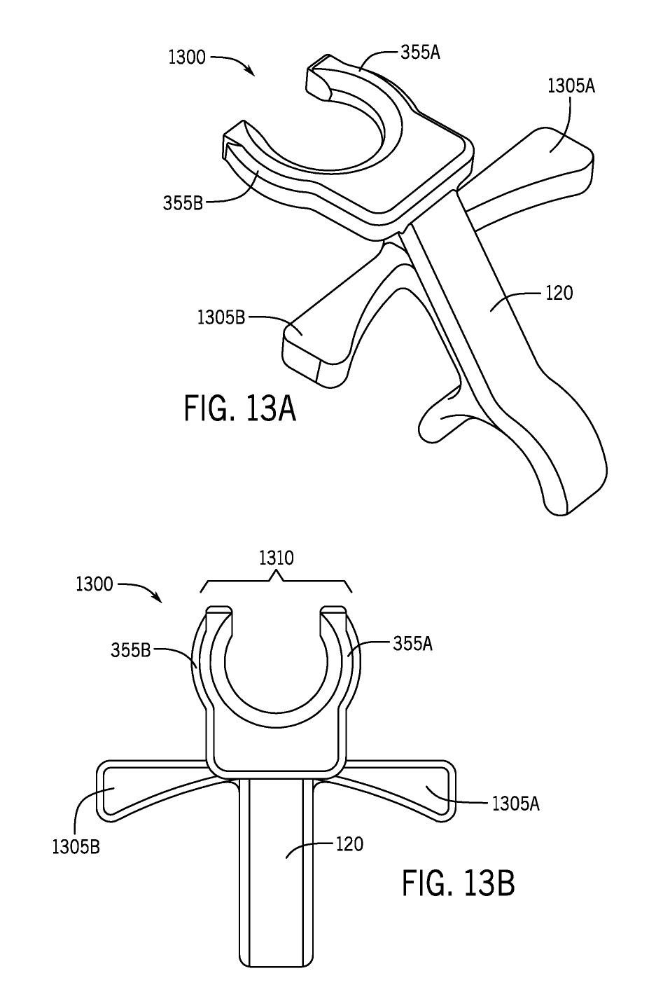

Example 9: Drop Dispenser for Multi-Use Container. FIGS. 13A and 13B illustrate a related embodiment 1300 of a passive device, structured to accommodate a multi-use container of liquid between the prongs 355A, 355B of a pitchfork portion 1310 of the device 1300 and that also features cross stabilizers or wing portions 1305A, 1305B. The cross-stabilizers or wing portions are affixed to the arm to extend in a direction that is simultaneously transverse to the arm and to a plane (not shown) passing through the arm and through the axis of the bore of the holding portion. The cross stabilizers 1305A, 1305B aid a user in stabilizing the device 1300 against another surface, such as a portion of the head (for example, forehead) of the user. This enables the user to achieve better spatial orientation of the device 1300 with one hand while, at the same time, also squeezing the multi-use container of liquid supported by and retained between the prongs 355A, 355B, with the same hand. This leaves the user's other hand available to hold open the eye to facilitate targeted delivery of the desired amount of fluid from the container (for example, a predetermined number of drops). This type of configuration may be beneficial for user's that have tremors or other motor control issues that benefit from the additional fixation point.

The shape, dimensions, angular orientation—generally, configuration - of the cross stabilizers in relation to that of the arm 120 may be arranged in various formats, including, but not limited, to having only one cross stabilizer (that is, a cross stabilizer on only one side of the arm 120). It should also be noted that, generally, any of related embodiments disclosed herein—where active, passive, or configured for use with a single-used container(s) of liquid—may be equipped with at least one of the cross stabilizers 1305A, 1305B.

### Active Embodiments and Methods of Operation of Same

The idea behind complementing an array or kit or set of passive fluid-dispenser holders with embodiments of active devices is to enable a user of the kit to at least train himself to deliver drops of fluid into his eye(s) based on operational feedback provided by the active device in terms of optical data representing circumstances of drop(s) delivery (such as timing, success of delivery). Alternatively or in addition, the optical data collected with the active device in an unbiased fashion (and without human interference) can be used by the observing clinician for the purposes of adjusting the type of treatment to be used (for example drops versus laser or surgery for glaucoma) or the addition or subtraction of a drop medication to the patient's treatment regimen or at least one of the dose and schedule of patient's (user's) treatment, in an exact and systematic fashion to answer questions relevant to the success of the patient's treatment and evaluation of outcomes of such treatment.

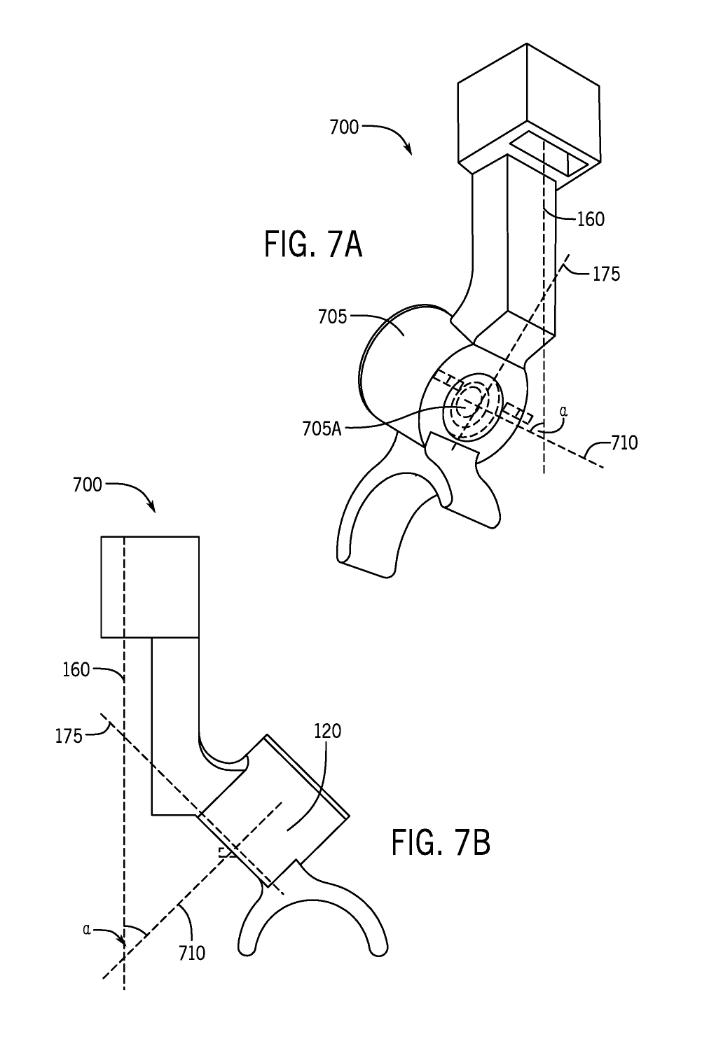

Example 10: Drop Dispenser for Single-Use Container. To this end, and referring to FIGS. 7A, 7B and 8A, 8B, 8C, embodiments of an active device may be substantially structurally similar in dimensions and size to their counterpart passive devices. FIGS. 7A, 7B, for example, show an embodiment 700 of an active device that is structured to accommodate single-use containers of liquid and that is substantially structurally similar to that of the passive device 100 of FIGS. 1A and 2A. The active device 700, however, is additionally equipped with a data-recording and/or processing unit 705 that includes an optical system 705A, disposed at the arm 120 between its first and second ends. The optical system 705A has a field-of-view (FOV) with a FOV axis 710. Such FOV covers a region of interest (ROI) 170 around a point on the first axis 160 in a plane transverse to the first axis 160, where the FOV axis 710 intersects the first axis 160 at the ROI 170. More specifically, as shown, the FOV axis 710 intersects the first axis 160 at an acute angle a, where the acute angle a is an internal angle of a triangle defined by the first axis 160, the second axis 175, and the FOV axis 710. By analogy with the embodiment 100, the element 122 (as shown - a shelf or protrusion from the body of the processing unit 705 represents the optional indicia of location, which in operation of the device signifies the level at which the tip of the nozzle of the used liquid container should be positioned when the container is retained in the device.

Example 11: Drop Dispenser for Single-Use Container. FIGS. 8A, 8B, and 8C show an active device 800 structured to accommodate multi-use containers of liquid, which is substantially structurally similar to the passive device 600 shown in FIGS. 6A and 6B. Similar to the active device structured to accommodate single-use containers of liquid, the active device 800 is equipped with an active unit 805 including an optical system 805A, disposed at the arm 120 between its first and second ends. The optical system 805A has a field-of-view (FOV) with a FOV axis 710, the FOV covering a region of interest (ROI) 170 around a point on the first axis 160 in a plane transverse to the first axis 160, and where the FOV axis 710 intersects the first axis 160 at the ROI 170. More specifically, the FOV axis 710 intersects the first axis 160 at an acute angle a, where the acute angle a is an internal angle of a triangle defined by the first axis 160, the second axis 175, and the FOV axis 710.

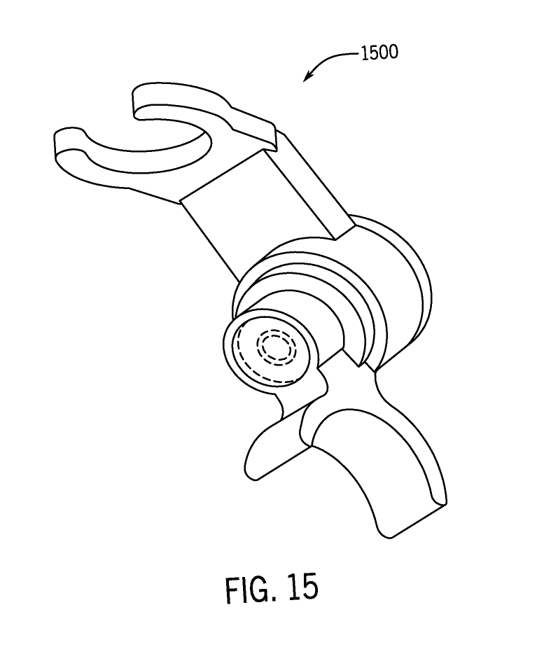

Depending on the specifics of use, each previously described embodiment of a passive device may be appropriately modified to be transformed into or formatted as an active device. For example, referring to FIG. 15, an active device embodiment 1500 structured to accommodate multi-use containers of liquid is configured substantially structurally similar to the embodiment 300 of the passive device shown in FIGS. 3, 4A, 4B, 4C, and 5.

Generally, the active unit of a given embodiment (such as the active units 705, 805) and an optical system 705A, 805A of such unit can be configured substantially equivalently for both an active device structured to accommodate single-use containers of liquid and an active device structured to accommodate multi-use containers of liquid. In related embodiments, the active units can be connected, via electrically-conducting members or otherwise, to a local or remote programmable processor, which is in operable cooperation with tangible non-transient storage medium. The processor in such case is configured to acquire optical data collected by the optical system 705 representing an image of a scene present at a given time at the ROI 170. These data can then be analyzed to determine if a liquid drop expelled from the nozzle (285 of the single-use container, or 460 of the multi-use container, affixed in the body 140 is successfully delivered to the ROI 170).

Alternatively or in addition, the optical system 705A, 805A could be augmented with a sensor system that contains a communications function. This sensor system could be disposed in a housing of the active unit 705, 805 and/or paired with a computer-based application to perform various functions. This computer-based application could, for example, be downloaded onto a smartphone or similar device. The sensor system could communicate with the application to indicate at which times the active device is being employed. If the active device is not being employed according to the specified scheduled, the application would alert the user via their smartphone to use the device to administer the missed drops after a scheduled administration is not sensed.

If a particular user's specific needs so demand, each of the embodiments described herein may be structurally adjusted to fit such specific needs. This adjustability may be embodied in the device itself (for example, at least one of the length of the arm 120 and the angle of the arm 120 with respect the foot or base 115, may be adjustable, via an appropriate telescopic mechanism and/or hinge)

Alternatively, or additionally, the customization of a specific embodiment may be achieved by fitting differently-sized devices to the particular user to determine which suits the needs of the user most effectively. Such fitting could be performed in a number of different ways, for example, it could be accomplished through input of the parameters of a particular liquid container being used into a database (which, with the use of a programmable processor would match the container, based on its dimension, to the most appropriate embodiment of the device). The fitting could also be done by office personnel and could involve the user trying different devices to determine which one works best for the user's particular needs. The process of fitting could also involve measuring the facial feature (such as the height of the bridge of the nose) of the user, and selecting the device based at least in part on the results of such measurement. Various device embodiments are optionally sizable, meaning that their dimensions can be individualized and that they are designed to pursue the variability of different lengths of liquid container tips, and/or distance of the tip to the ROI, and/or the anatomy of the user, and/or problems individual to a user (e.g., tremor, stiff neck and cannot lean back, keeps touching eye and needs offset from eye, or closes eye and drop lands on upper lid or lower lid).

The use of either of an active device of the invention or a passive device of the invention, or a set of such devices is intended to include at least some of the following steps: using the active device with a liquid-containing capsule affixed in a bore/opening thereof to deliver a series of liquid drops; monitoring a data set representing a delivery of the series of liquid drops supplied by the active device with the capsule in it; making adjustments to a user's delivery technique based on assessment of the data set; and initiating a use of a passive device with a liquid-containing capsule affixed in a bore thereof to deliver liquid drops when the data set indicates that the user's delivery technique is successful in delivering liquid drops to a region of interest as intended. The “intent” of delivery, determining its success, may include successful targeting of the ROI with a liquid drop and/or targeted delivery of such drop according to a pre-determined schedule. While this example of a method of use of a set of devices illustrates a progression from using an active device to using a passive device, it should be noted that the progression of use of the system of devices could be from use of a passive device to that of an active device. As an example, a user may be initially using a passive device, but unsuccessfully in that the user fails to administer the drops as intended, in which case the user may be directed to use an active device with a sensor, feedback capable system for active monitoring of drop delivery and/or reporting of the results of such monitoring. In this specific example, the collection of feedback data is prompted by lack of success of using the “passive” device on the user's part.

In some users that start with the passive device and do not experience difficulties with delivery and compliance, it may not be necessary for them to use an active device and the passive device may suffice.

The active monitoring device can also be used to assist drop delivery and determine if a drops is being used successfully. If delivery is not successful, even with the assist device, it may be determined that adding additional drops would not help since they are not getting into the eye not because they are not working. This information can then be used to try and train the individual on proper drop use and not to add more drops. It can also lead to the determination that an alternative treatment methods, say laser or surgery for glaucoma patients is preferred to additional drops. For physicians and insurance companies, this can reduce waste, and insure that the treatment is individually targeted to the patient based on the patients individual needs to result in better outcomes and less waste.

The active device has been described as including a processor controlled by instructions stored in a memory. The memory may be random access memory (RAM), read-only memory (ROM), flash memory or any other memory, or combination thereof, suitable for storing control software or other instructions and data. Those skilled in the art should readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while a portion of the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

While the invention is described through the above-described exemplary embodiments, it will be understood by those of ordinary skill in the art that modifications to, and variations of, the illustrated embodiments may be made without departing from the inventive concepts disclosed herein. For example, although some of the embodiments are described in connection with various illustrative structures, one skilled in the art will recognize that the system may be embodied using a variety of different structures. Examples of related embodiments of individual devices are illustrated in Appendix A. Modifications of embodiments may include an added contraption configured to compress the bottle automatically or electronically to improve drops delivery, or which is only activated to deliver the drops when the device is placed in the correct position. Modifications may further include, for specific embodiments configured to be used with a ROI that includes an eye of the user, different structures of the arm or arm of a device, a light source added to the optical system and having an illumination zone that includes a tip of the single use liquid drop dispenser and the eye of the user located below the rectangular cavity when the arcuate base of the first end is positioned above the user's nose root; and/or a compressor unit configured to compress a liquid holding container against a side of the arm; and/or a sensor system to communicate with a computer-based application to indicate at which times the liquid drop delivery device is being employed; and/or a the computer code governing the processor to generate data to inform the user that a scheduled drop was missed.

The scope of the invention includes, for example, a set of devices configured to retain a dispenser of liquid, where the set includes a first device having (a) a base with a top side and a bottom side, the base having first and second base ends; (b) an arm extending from the top side of the base along a first axis and attached to the base at a first end of the arm at a point that is separated from a first plane passing through the first and second base ends; and (c) a holder portion with an opening formed therethrough, the opening having a second axis that is inclined with respect to the first axis, a cross-section of the opening in a second plane that is transverse to the second axis being one of (i) a cross-section containing a right angle, and (ii) as a cross-section in which the opening defines a curve (in a specific case - a curve having a constant radius of curvature). Such set may additionally include a unit containing an optical system with a field-of-view, the unit being cooperated with the arm to orient the FOV to cover a tip of a nozzle of the dispenser when the dispenser is retained in the opening and oriented with the tip pointing towards the first plane. In one embodiment, the set is configured to meet at least one of the following conditions: (i) the base is arcuately shaped and dimensioned to fit above and in contact with a user's nose bridge; (ii) the base is curved in a plane that contains the second axis, while a cross-section of the opening defined in a plane perpendicular to the second axis has one of (a) a curvature in a plane perpendicular to the bore axis, and (b) a closed perimeter; (iii) at least one of a length of the arm and an inclination of the arm with respect to the base is adjustable; (iv) the first device further includes a wing portion affixed to the arm at a point between holding portion and the base and extending from the arm along an axis that is transverse to a reference plane, the reference plane containing the bore axis and passing through the arm; and (v) the first device further includes a platform, repositionably disposed substantially parallel to a cross-section of the opening, and a return mechanism configured to apply a bias force to the platform in a direction of the opening The first device in the set may additionally contain an extension, protruding from the holder portion towards the first plane to form a gap between the arm and the extension, (the gap configured to accept a first finger of a user when the dispenser is retained in the opening and, while a second finger of the user is in contact with a surface of the dispenser, to allow to squeeze the dispenser between the extension and the second finger). The extension may carry at least one tongue protruding from the extension on a surface facing away from the gap, and wherein the cross-section of the opening is a rectangular cross-section dimensioned to retain a tail portion of a single-use dispenser filled with eye drops. The set may include a second device configured substantially equivalently to the first device, wherein dimensions of at least one component of the second device appropriately differ from those of a corresponding component of the first device to accommodate different physical characteristics of a user choosing a device from the set (i) to retain the dispenser in the opening and position the base on a bridge of a nose of the user with a tip of a nozzle of the dispenser being separated from an eye of the user by about 10 mm, and (ii) to successfully deliver a drop of the liquid from the nozzle to a surface of an eye while the device is so positioned.

The scope of the invention also covers a system configured to retain a dispenser of liquid, where the system contains a holder portion with an opening configured to retain and fixatedly support the dispenser of liquid, the dispenser having a nozzle, and a body extension that is attached to and stretches forth from the holder portion and that is configured, in operation, to contact a user to support the dispenser at a separation distance from the user. The dimensions of various components of the system can vary (while generally retaining their structural characteristics) to accommodate differences in anatomies of users of the system and dispensers of different lengths. In a specific case, the system may include a first device that has such body extension, and in which case the body extension contains a base with a top side and a bottom side, the base having first and second base ends, and an arm extending from the top side of the base along a first axis and attached to the base at a first end of the arm at a point that is separated from a first plane passing through the first and second base ends. The first device also includes the above-described holder portion with an opening formed therethrough, the opening having a second axis that is inclined with respect to the first axis, a cross-section of the opening in a second plane that is transverse to the second axis being one of (i) a cross-section containing a right angle, and (ii) as a cross-section in which the opening defines a curve. IN a related embodiment, the system additionally comprises a second device that is substantially structurally equivalent to the first device and that, in addition, contains at its arm, a data-recording unit containing an optical system with a FOV defined to cover and include a tip of the nozzle when the dispenser is retained at the opening

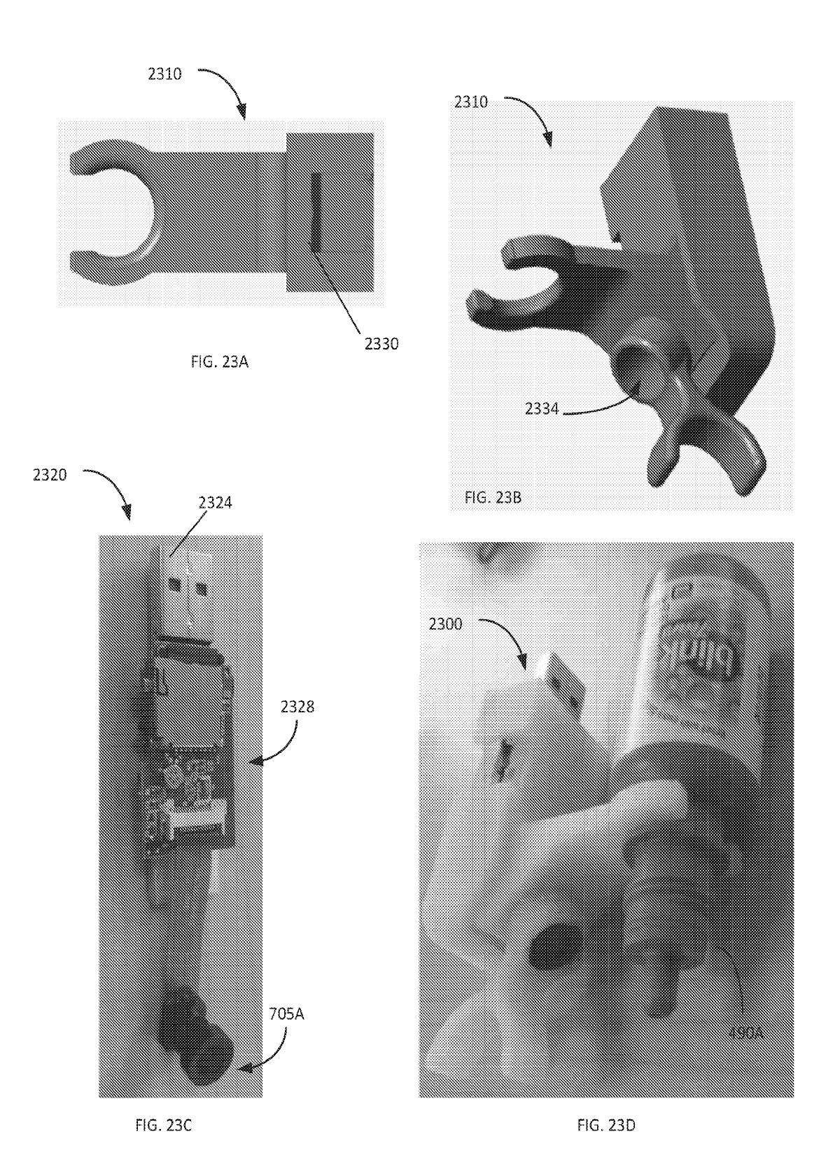

One non-limiting modification includes a composite implementation 2300 of the active device shown in FIGS. 23A, 23B, 23C, and 23D, which includes a housing shell 2310 (shown in FIGS. 23A and 23B in top and perspective views, respectively) and the “filler” portion 2320 that includes active components (at least such as, for example, electronic circuitry 2324 appended with the USB port 2328 (or another data port conforming to the chosen standard) on one end and with the optical portion 705A on the other end, as shown - through the ribbon cable. Prior to operation, the portions 2310, 2320 are combined by appropriately inserting the filler portion 2320 into the housing 2310 switch that the data port 2324 outwardly protrudes through the correspondingly-dimensioned slit 2330 while the optical system has unblocked FOV through the cylindrical opening 2334 of the shell 2310 (as shown in FIG. 16D, in which the assembled active device 2300 is shown retaining the liquid container 490A in the opening of the pitchfork portion).

Accordingly, the invention should not be viewed as being limited to the disclosed embodiment(s).

### APPENDIX

Here, the results of one of several empirical studies are summarized to highlight the unmet need(s) related to the use of liquid drop dispensers (such as lack of ability to evaluate and trains the user to correctly and efficiently deliver a liquid drop from the dispenser to the ROI when the currently-used in the related art devices and methods for such delivery are being used in at-home environment and /or without supervision) and satisfaction of such needs with the use of an embodiment of the invention.

### Evaluation of At-Home Eye Drop Delivery in Post-Cataract Surgery Patients

It is well recognized that while patient adherence to a prescribed ophthalmic regimen is problematic yet data assessing eye drop instillation outside of the clinic is limited.

Cataracts affect approximately 17% of Americans 40 or older and this number increases to more than 65% by age 80. Consequently, cataract surgery is one of the most frequently performed outpatient procedures in medicine, with over 3 million procedures per year in the USA alone. After the surgery, physicians typically prescribe antibiotic eye drops to prevent infection and anti-inflammatory eye drops to help reduce inflammation and speed visual recovery. Failure to adhere to the post-surgical eye drop regimen can lead to complications such as severe inflammatory reactions, macular edema, and endophthalmitis which can cause vision loss and intraocular scarring. Additionally, improper delivery (for example, with the tip contacting and pressing on the cornea) can result in wound distortion and intraocular infections. Another significant issue with eye drops that is consistently reported by physicians and patients is problems with drop adherence. Studies have shown that over 90% of patients may not be using their eye drop medications correctly, though only 31% of patients report having difficulty instilling their eyedrops. Not only does incorrect dosing have the potential to impair the visual recovery process following the surgery and increase the risk of inflammation and/or infection, but incorrect perceived or actual deviations from the prescribed regimen can also restrict a physician's ability to determine the optimal therapeutic regimen. Furthermore, cases of poor drop-delivery techniques, which require additional drop applications to be compensated, can drive up health costs if an early refill of the liquid medication is needed.

The purpose of this study was to directly assess at-home patient adherence with prescribed eye drops in post-cataract surgery patients. Poor adherence, whether intentional or unintentional, is most likely due to the discrepancy between perceived versus actual administration of eye drops where subjects believe they are performing better than what is actually occurring. Thus we hypothesize that the perceived drops dispensed and drops landing in the eye will be significantly different from both the actual dosing and the prescribed regimen.

### Methods

Inclusion/exclusion criteria: An observational case study was conducted at a single site with twenty--seven post-cataract surgery patients (age 58-92; mean age 71) taking prescription eye drops. Only those who could self-consent were enrolled in the study. All patients reported their drop usage in a daily log and used an embodiment of an active (imaging) device to record their prescribed eye drop administration for one week. Video data from the recording device were further processed.

The average number of time points recorded per patient was 33±3 (range 25-42). In total, 881 videos were recorded and analyzed. All subjects in the trial signed informed consent forms and Institutional Review Board approval for this study was obtained from the Lee Memorial Health System. It is certified that all compliance requirements were adhered to and all applicable institutional and governmental regulations concerning the ethical use of human volunteers were followed during this research.

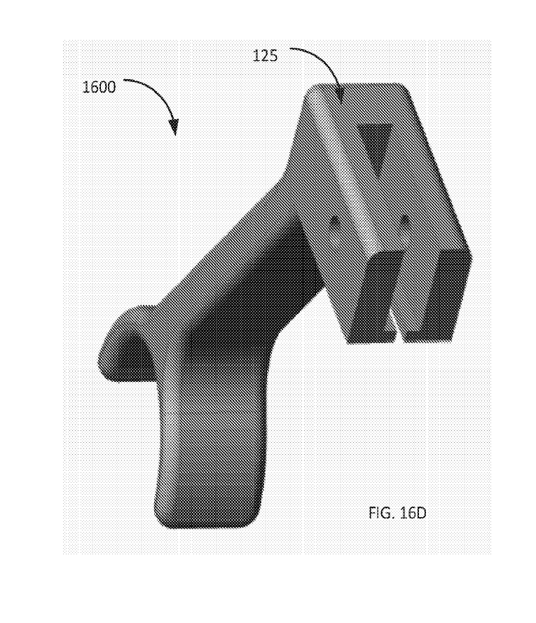

Study Design: This was a non-randomized prospective pilot study. Twenty-seven participants were instructed on use of an embodiment of the novel imaging device, referred to herein as EDAM. The active device, the embodiment of which was discussed in U.S. patent application Ser. No. 14/438,716 (and is incorporated herein by reference in its entirety), is shown in frame A of FIG. 17. The embodiment includes a camera and a recorder. The camera piece contains a camera which is flanked by two LED lights. There are two arms and a base just below the camera that attach to most eye drop bottles. The recorder powers the camera and LED lights and stores recorded videos for later review (FIG. 17 frames, B, C, D). Following the training, the EDAM device was used for one week with all prescribed eye drops adhering to the regimen described above. Further, patients were given a log where they noted how many landed in the eye, outside the eye, or half in and half out for each administration over the course of the week. The captured videos were analyzed by the reading center to determine the patients' eye drop use and delivery. Comparisons between subjects' perceived drop usage was compared to what was observed by the reading center (actual usage). Drops recorded as half in and half out were give a score of 0.5. Videos absent for a particular time point were considered a missing event and therefore counted as a 0 towards their total drop usage.

Statistical Analysis: The number of drops dispensed, drops in, and drops half in and half out were compared between the subject (perceived), the reading center (actual), and the prescribed regimen. Particularly the actual treatment (drops in/prescribed), intention to treat (drops dispensed/prescribed regimen), and success rate (drops in/drops dispensed) were calculated and converted to percentages. Percentages were used because the prescribed regimen varied slightly between patients depending on the time of enrollment i.e. patients enrolled in the afternoon so any drops administered prior were excluded from the prescribed regimen. Percent absolute variations were calculated by subtracting each value from 100% and converting each difference into the absolute value. By comparing absolute variations, the deviation from the prescribed regimen is better visualized as overall means may misrepresent adherence rates since upper and lower values negate each other. Paired t-tests were used to assess significance in all analyses (GraphPad Prism 4). A power analysis to determine sample size was not completed. Further, the small sample size did not allow for the assessment of covariates such as age, sex, or education on drop delivery in this pilot study. Values given are mean and standard deviation.

### Results

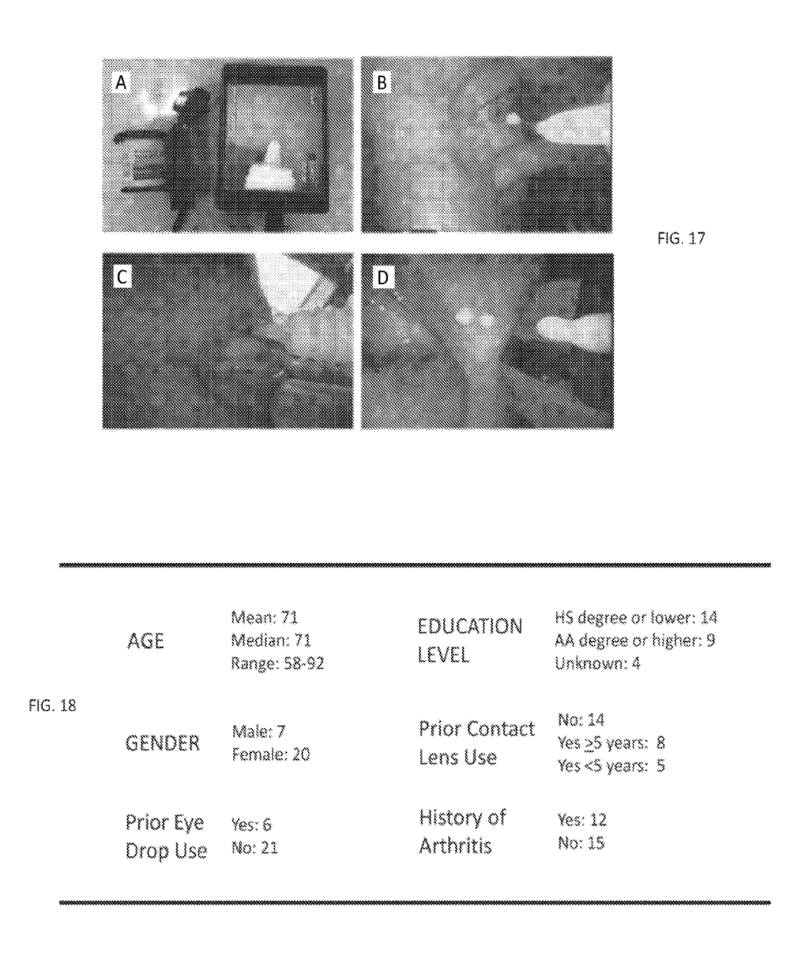

Patients who underwent cataract surgery from a single private practice were enrolled in a study in which their eye drop administrations were monitored using the EDAM device. Thirty-two subjects were enrolled; however, five subjects opted out of the study after enrollment. Twenty-seven patients completed the study and their demographic information is provided in Table of FIG. 18. During enrollment, all subjects were asked optional questions regarding their experience with prescription eye drops, education level, history of contact lens use, and history of arthritis.

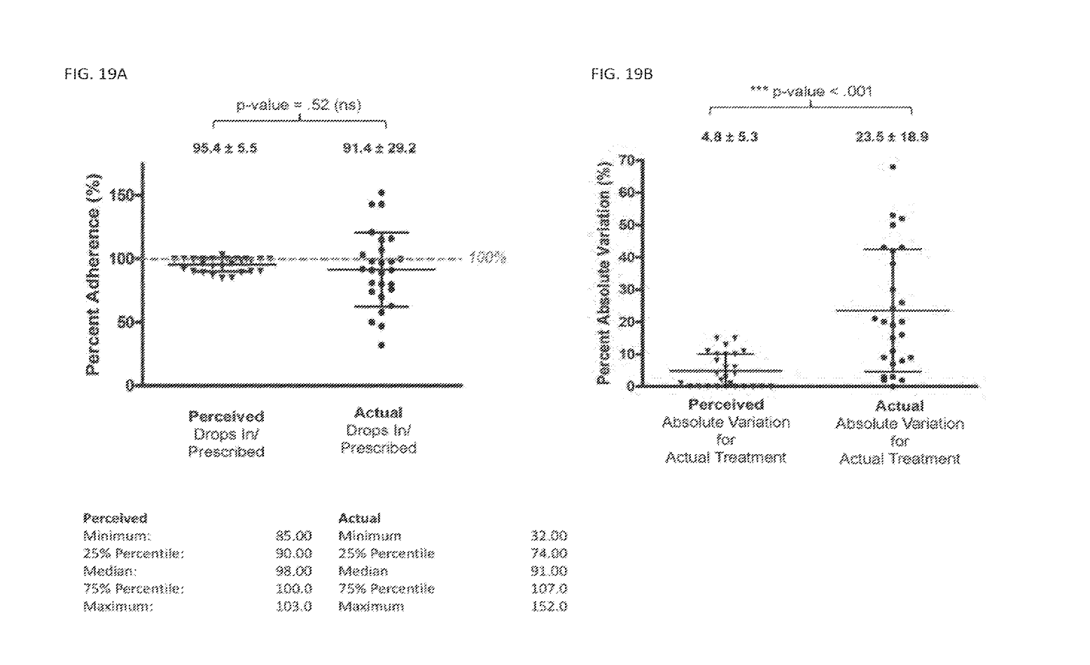

Subjects' perception (Perceived) of their drop administration was determined from the supplied log they filled out during the study period and compared to the videos captured on the EDAM device (Actual). Examples of images recorded are shown in frames B, C, and D of FIG. 17. The actual treatment, number of drops in relative to the prescribed regimen, was calculated and results are shown in FIG. 19A. On average, subjects perceived that they successfully administered 95.4%±5.5 (range 85%-103%) of their drops compared to 91.4%±. 29.2 (range 32%-152%) observed from videos captured on the EDAM device. Although the average actual treatment was not significant (p=0.52), the range for perceived number of drops in/prescribed regimen and actual number of drops in/prescribed regimen differ greatly. Clinically, this is the most important data. If a patient only achieves a successful drop delivery of 30%, that is important. Knowing that this is balanced out by another patient that is wasting their drops, is not clinically useful. To achieve a better metric for the variation from the prescribed regimen, the percent average absolute differences were calculated. This summary statistic highlights the variability from the prescribed regimen and was calculated for the intention to treat, as well as for the success rate. Hence comparing the percent absolute deviation for actual treatment (FIG. 19B), the perceived absolute variation is 4.8%±5.3 and the actual absolute variation is 23.5%±18.9 (p<0.001).

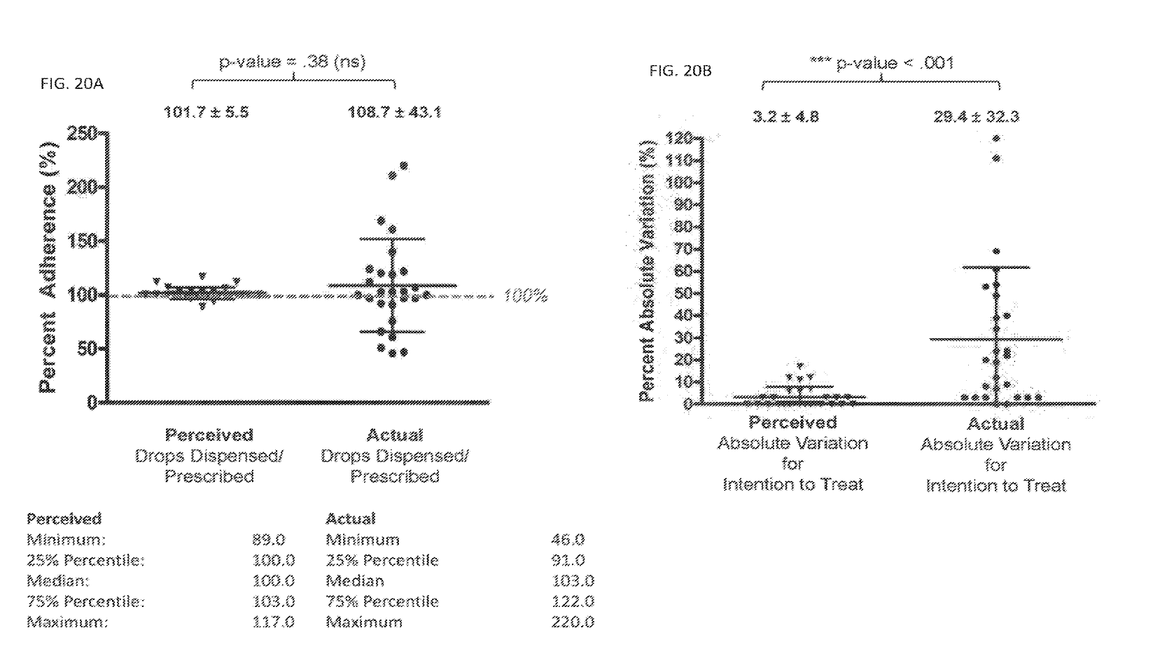

Comparing the intention to treat (FIG. 20A), number of drops dispensed divided by the prescribed regimen; subjects' perceived they dispensed 101.7%±5.5 (range 89%-117%) of the drops prescribed compared to what they actually did 108.7%±43.1 (range 46%-220%). Similar to actual treatment, the average intention to treat for both data sets are not significant (p=.38) yet in the actual data set captured by the EDAM there is again far more variability than in the perceived data set. Comparing the percent absolute variation of intention to treat (FIG. 20B), subjects' perceive they deviate from the prescribed regimen by 3.2%±4.8 when they actually deviate 29.4%±32.3 (p<.001).

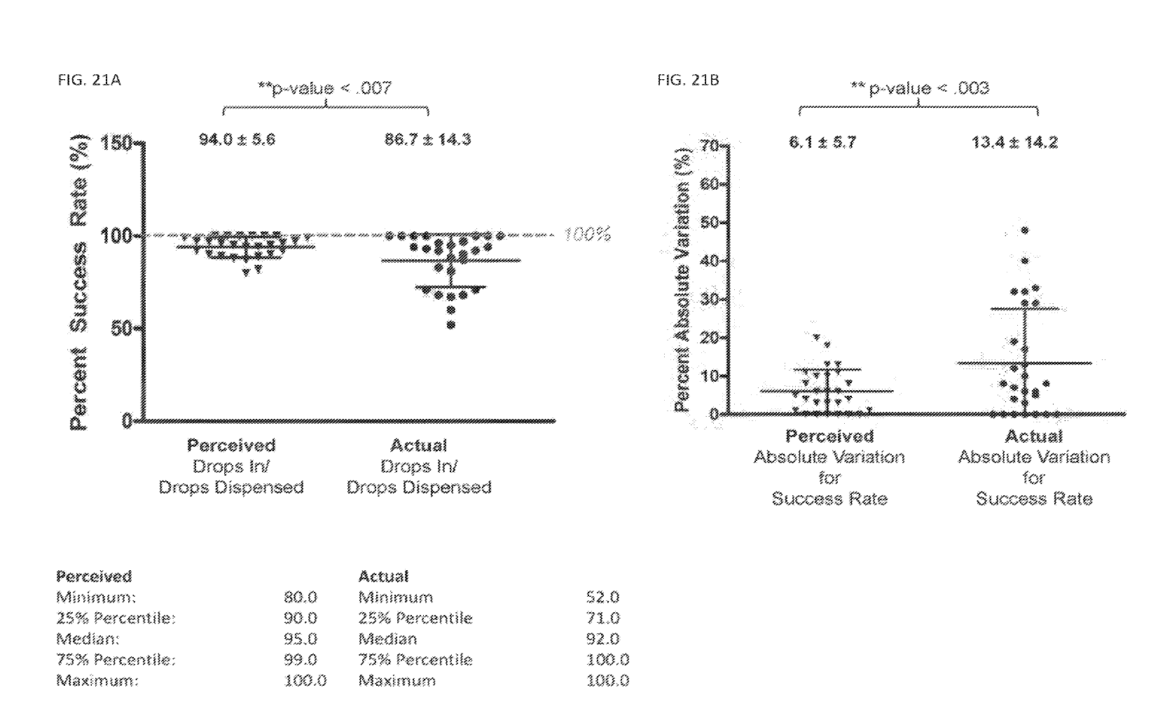

FIGS. 21A, 21B show the perceived and actual success rate, number of drops in relative to the number of drops dispensed. Subjects' perceived that 94.7%±5.6 (range 80%-100%) of the drops dispensed were successfully administered into the eye; however, this is significantly different (p<0.007) than their actual success rate of 86.7%±14.3 (range 52%-100%). Further, the absolute variation from the success rate was also significantly different between perceived and actual (p <0.003) with averages of 6.1% ±5.7 and 13.4% ±14.2 respectively.

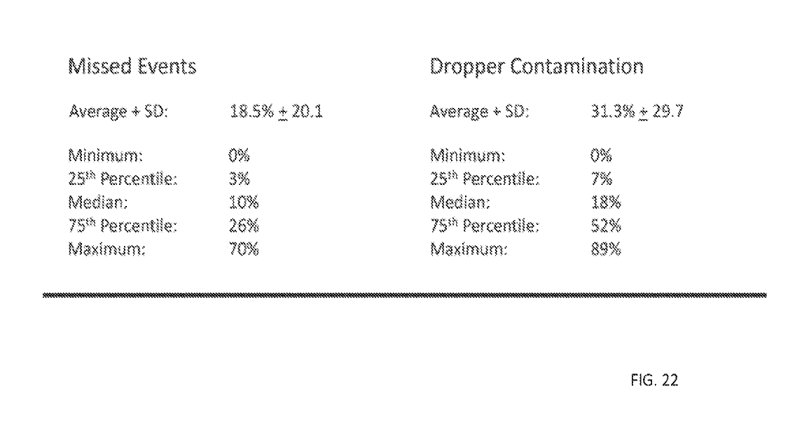

The number of missed events (i.e. no video for a particular time point and presumably no drops dispensed) and the number of times the dropper tip came in contact with the cornea, eyelashes, or skin were noted during video analysis (see Table of FIG. 22). On average, subjects failed to administer their drops 18.5%±20.1 (range 0%-70%). Only four subjects administered all of their scheduled eye drops throughout the course of the study. Contamination is also a significant problem as roughly one/third (31.3%±29.7) of each administration resulted in contamination of the dropper tip. In total, 89% (23/27) of the subjects contaminated their dropper tip at least once over the duration of the study.

### Discussion

This study is the first to directly measure adherence with postoperative medications following cataract surgery while at patients' homes. Overall, our results are consistent with previous findings that patient reports of eye drop regimen adherence are significantly different from their actual adherence. In the early 1980s, Kass et al interviewed 141 patients on their eye drop adherence and then observed their administration. They reported that 83% of patients instilled one drop per application yet during observation over 48% dispensed two or more drops. Similarly, Stone et al questioned 139 patients on their topical medication usage and subsequently video recorded their eye drop instillation using two different bottles. They reported that over 90% of patients believe they do not have difficulty putting in their drops; however, less than 31% of patients administered a single drop without contaminating the drop tip. In our observations, we report similar discrepancies between what patients perceive compared to what is recorded on the EDAM. For instance, patients reported that they applied the correct number of drops and only deviated from the prescribed regimen by 5% but in reality they deviated by almost 24%. Comparing the intention to treat yielded similar results where patients believed they are dispensing the appropriate amount but after video analysis it is disclosed in FIG. 3B that they varied from the prescribed regimen by almost 30%. Further, 5 (18.5%) subjects over-dispensed their drops by greater than 30% making them at risk of running out of their medication prior to the end of their prescribed regimen. While it is clear that some patients are having difficulties instilling their ophthalmic drops, this is not the case for every patient. For example, twelve (44.4%) of the twenty-seven patients had an adherence rate of at least 80% or higher with regard to actual treatment, intention to treat, and success rate. Therefore, to improve overall drop delivery and compliance, it is critical to identify those patients who are having problems to allow for retraining or utilization of alternative postoperative drug delivery options.

Topical antibiotic, steroid, and nonsteroidal eye drops are commonly prescribed following cataract surgery, but if a patient is non-adherent or incorrectly applies them in a way that disrupts or opens the incision they may increase their risk of post-operative infection(s). Endophthalmitis following cataract surgery is a rare (approximately 1 in 800 cases) but serious occurrence which can result in vision loss. Failure to achieve prescribed antibiotic, steroidal, and non-steroidal eye drops delivery to the eye can increase the risk of inflammation, pain, scarring, and cystoid macular edema which can delay and/or impair visual recovery. Unwanted contact with the lids and cornea can cause irritation, wound gape, and potentially increase the risk of infection and other problems. Our data indicates that drop delivery is a significant problem following cataract surgery. How best to manage this risk has yet to be determined. Attempts have been made to address issues associated with drop delivery and non-adherence, including phone apps, automated voice and text alerts, retraining, physical devices for assisting with drop placement and bottle squeezing, and altering the color and/or shape of the dropper bottle to increase usability.

### Conclusion and Relevance

It has long been known that variation in drop dispensing and delivery with the prescribed eye drop regimens is an issue; however, the depth of the problem has never been directly determined at home following cataract surgery. The results of the above-presented study indicate that lack of eye drop delivery is a ubiquitous problem and affects anyone prescribed ophthalmic drops, although it does affect some more than others. When this is compounded with general problems of adherence (where forgetfulness is the most commonly cited issue, resulting in missing a drop administration), it is easy to appreciate the combined significance of both of these problems to the delivery of health care to patients. Currently, physicians must evaluate the state of the disease of their patients and the effect the prescribed regimen based on a patients presentation and self-reported (or perceived), adherence rather than the actual objective data. While assumptions about therapeutic effects are not ideal, the assumptions have been the only option up to-date because direct, objective data have not been available. Through the use of the embodiment of the invention (EDAM), a significant discrepancy between perceived and actual drop dispensing and delivery, by the patient's themselves and without supervision. The results highlight the necessity of collecting accurate data of not only adherence but of eye drop applications from patients outside of the clinical environment. Such information is especially pertinent for physicians to better understand and positively affect clinical outcomes for each and every patient or for those interested in ensuring proper delivery of topical medications.

1. A set of devices configured to retain a dispenser of liquid, the set comprising:
an active device and a passive device, each of the active and passive devices comprising:
a base with a top side and a bottom side, the base having first and second base ends;
a holder body having first and second sides and an opening formed therethrough along a first axis from the first side to the second side, the opening being sized to retain the dispenser having a nozzle,
an arm elongated along a second axis, the arm having a first end affixed to the holder body and a second end affixed to the base;
and
wherein the active device further comprises, at its arm, a data-recording unit containing an optical system with a field-of-view (FOV) defined to cover and include a tip of the nozzle, when the dispenser is retained at the opening.

2. The set of devices according to claim 1, wherein one or more of the following conditions is satisfied:
(i) wherein, for every device in the set:
the base is arcuately shaped and dimensioned to fit above and in contact with a user's nose bridge;
the device is dimensioned such that, when the base is on a user's nose bridge, the first axis passes through or around an eye of the user when; and
the opening has a cross-section in a plane transverse to the first axis, the cross-section being one of (i) a rectangular cross-section and (ii) as a cross-section in which the opening defines a curve;
(ii) wherein at least one of a length of the arm and an inclination of the arm with respect to the base is adjustable to position the first axis to pass through or around an area of a user's eye once the base is disposed on a user's nose bridge;.
(iii) the second axis is inclined with respect to the first axis.

3. The set of devices according to claim 1, wherein one or more of the following is satisfied:
(i) at least one device in the set comprises a wing portion affixed to the arm at a point between the holding body and the base and extending from the arm along an axis that is transverse to a reference plane, the reference plane containing the first and second axes;
and
(ii) for each device in the set, the base is curved in a plane containing the first axis, while a cross-section of the opening, defined in a plane perpendicular to the first axis, has one of (a) a curvature in a plane perpendicular to the bore axis, and (b) a closed perimeter;
and
(iii) wherein for each device in a set the bottom side is curved with a center of curvature located in a plane containing both the first and second axes, the center of curvature being separated from the first end by a distance exceeding a length of the arm;
and
(iv) wherein the holding body is a cuboid.

4. The set of devices according to claim 1, wherein a structural configuration of the active device and a structural configuration of the passive delivery device are substantially the same with an exception of the data-recording unit present at the arm of the active device.

5. The set of devices according to claim 1, wherein the active device further comprises one or more of
(i) a programmable computer-readable processor in operable cooperation with tangible non-transient storage medium, the processor configured to acquire optical data that have been collected by the data-recording unit and that represent a scene at the target region; and
(ii) a sensor system configured to wirelessly communicate with a programmable electronic circuitry to produce a record of time schedule of actual use of the active device, that has been equipped with the dispenser, for drop delivery into the target region.

6. The set of devices according to claim 1, wherein an optical axis of the optical system intersects the first axis at the target region.

7. The set of devices according to claim 6, wherein the optical axis intersects the first axis at an acute angle, the acute angle being an internal angle of a triangle defined by the first axis, second axis, and optical axes.

8. The set of devices according to claim 1,
wherein each of the active and passive devices dimensioned such that, when the dispenser is retained in the opening with the nozzle directed along the first axis and away from the holder body, a target region for delivery of a drop of the liquid contained in the dispenser is defined in a first plane that is parallel to a plane passing through the first and second base ends; and
wherein the FOV is defined to cover and include the target region.

9. The set of devices according to claim 8, wherein the first axes passes through the target region.

10. A method for using a set of devices configured to retain a dispenser of liquid, the set including an active device and a passive device,
wherein each of the active and passive devices contains
a base having a top side, a bottom side, and first and second base ends;
a arm extending from the top side of the base at a first end of the arm;
a holder portion with an opening therethrough, the opening having an opening axis and sized to retain a liquid drop container having a nozzle,
wherein the active device additionally includes, at its arm, a data-recording unit containing
an optical system with a field-of-view (FOV) defined to cover and include (i) a tip of the nozzle of the container retained in the bore, (ii) the target region, and (iii) a space separating the tip from the target region, and
an optical detector,
the data-recording unit configured to record images of a scene within the FOV;
the method comprising:
applying hand input to the active device to emit a liquid drop from the container retained by the active device in a direction of the target region while recording a series of image frames, each frame representing a corresponding position of the drop in the space;
monitoring, with the use of the data-recording unit, whether the drop landed in, partially in, or out of the target region;
and
using the passive device to deliver a liquid drop from the container retained therein to the target region.

11. The method according to claim 10, wherein a third plane tangential to a point at the top side and the second plane are parallel to one another.

12. The method according to claim 10, wherein the using includes applying hand input to the passive device to emit a liquid drop from the liquid drop container retained in by the passive device in the direction.

13. The method according to claim 10, wherein the using includes starting to use the passive device to deliver the liquid drop from the container retained therein when results of the monitoring indicate successful and repeatable delivery of the drop to the target region.

14. The method according to claim 10, wherein at least one of the following is satisfied:
(i) one or more of the applying, and using includes self-administering of the drop by the user without supervision;
(ii) the monitoring includes monitoring via a remote unit either by hardware download or wirelessly; and
(iii) any of the applying and using includes compressing the liquid container, that has been retained at the opening, against a lateral protrusion located between the holder portion and the base with one of (a) a finger, and (b) a level portion of the device hingedly attached to the holder portion while the base is positioned on a nose bridge of a user with the bottom side in contact with the nose bridge, and wherein the target region is the user's eye.

15. The method according to claim 10, wherein each of the applying and monitoring are carried out after the using.

16. The method according to claim 10, further comprising one or more of
(i) adjusting one or more of a position of the active device, an orientation of the active device, and the hand input based on results of the monitoring; and
(ii) inserting the liquid drop container in the opening of the holder portion to retain the container along the opening axis with the nozzle pointing towards the target region.

17. The method according to claim 16, wherein the inserting includes one of the following condition:
(i) retaining a flat tail portion of a single-use squeezable liquid drop container in said opening, the opening defining a hollow through the holder portion, the opening axis being completely surrounded by the holder portion in a cross-section that is transverse to the opening axis;
and
(ii) retaining a cylindrical neck of a multiple-use squeezable liquid drop container in said opening, wherein the opening is formed between first and second prongs of the holder portion, the first and second prongs extending transversely to the arm.

18. The method according to claim 17, wherein at least one of the top side and the art includes indicia of location, and wherein any of the retaining the flat tail portion and retaining the cylindrical neck includes positioning a tip of the nozzle substantially at a level of said indicia of location.

19. The method according to claim 10, further comprising one or more of:
(i) positioning any of the active and passive device on a nose bridge of a user while bringing a cross-stabilizer portion of the device in contact with a forehead of the user, wherein the cross-stabilizer portion extends transversely from the art between the holder portion and the base;
and
(ii) with a programmable electronic circuitry, generating a user-perceivable report containing data that represent whether the drop landed in, partially in, or out of the target region, and based at least in part on a figure of merit calculated from said data and representing success of a drop delivery to the target region the report, determining if a change of employing the active device to employing the passive device is appropriate.

20. The method according to claim 10, wherein, in each of the active and passive device, the holder portion is affixed to a second end of the arm such that when a corresponding device is in operational position with the liquid drop container retained at the opening, a target region for delivery of drops emitted from the nozzle is defined in a first plane that is parallel to a plane passing through the first and second base ends, the opening axis passing through the first plane near the target region.

21. A set of devices configured to retain a dispenser of liquid, the set comprising:
a first device having
a base with a top side and a bottom side, the base having first and second base ends;
an arm extending from the top side of the base along a first axis and attached to the base at a first end of the arm at a point that is separated from a first plane passing through the first and second base ends;
a holder portion with an opening formed therethrough, the opening having a second axis that is inclined with respect to the first axis, a cross-section of the opening in a second plane that is transverse to the second axis being one of (i) a cross-section containing a right angle, and (ii) as a cross-section in which the opening defines a curve.

22. The set according to claim 21, further comprising a unit containing an optical system with a field-of-view (FOV), said unit cooperated with the arm to orient the FOV to cover a tip of a nozzle of the dispenser when the dispenser is retained in the opening and oriented with the tip pointing towards the first plane.

23. The set according to claim 21, wherein at least one of the following conditions is satisfied:
(i) the base is arcuately shaped and dimensioned to fit above and in contact with a user's nose bridge;
(ii) the base is curved in a plane that contains the second axis, while a cross-section of the opening defined in a plane perpendicular to the second axis has one of (a) a curvature in a plane perpendicular to the bore axis, and (b) a closed perimeter;
(iii) at least one of a length of the arm and an inclination of the arm with respect to the base is adjustable;
(iv) the first device further comprises a wing portion affixed to the arm at a point between holding portion and the base and extending from the arm along an axis that is transverse to a reference plane, the reference plane containing the bore axis and passing through the arm;
(v) the first device further comprises a platform, repositionably disposed substantially parallel to a cross-section of the opening, and a return mechanism configured to apply a bias force to the platform in a direction of the opening

24. The set according to claim 21, wherein the device further comprises an extension, protruding from the holder portion towards the first plane to form a gap between the arm and the extension, the gap dimensioned to accept a first finger of a user when the dispenser is retained in the opening and while a second finger of the user is in contact with a surface of the dispenser to squeeze the dispenser between the extension and the second finger.

25. The set according to claim 24, wherein the extension carries at least one tongue protruding from the extension on a surface facing away from the gap, and wherein the cross-section of the opening is a rectangular cross-section dimensioned to retain a tail portion of a single-use dispenser filled with eye drops.

26. The set according to claim 21, further comprising a second device configured substantially equivalently to the first device, wherein dimensions of at least one component of the second device differ from those of a corresponding component of the first device to accommodate different physical characteristics of a user choosing a device from the set (i) to retain the dispenser in the opening and position the base on a bridge of a nose of the user with a tip of a nozzle of the dispenser being separated from an eye of the user by about 10 mm, and (ii) to successfully deliver a drop of the liquid from the nozzle to a surface of an eye while the device is so positioned.

27. A system configured to retain a dispenser of liquid, the system comprising:
a holder portion with an opening configured to retain and fixatedly support the dispenser of liquid, the dispenser having a nozzle;
and
a body extension that is attached to and stretches forth from the holder portion and that is configured, in operation, to contact a user to support the dispenser at a separation distance from the user,
wherein the system is variably dimensioned to accommodate differences in anatomies of users of the system and dispensers of different lengths.

28. The system according to claim 27, comprising
a first device including said body extension, the body extension containing
a base with a top side and a bottom side, the base having first and second base ends; and
an arm extending from the top side of the base along a first axis and attached to the base at a first end of the arm at a point that is separated from a first plane passing through the first and second base ends;
the device further including
the holder portion with an opening formed therethrough, the opening having a second axis that is inclined with respect to the first axis, a cross-section of the opening in a second plane that is transverse to the second axis being one of (i) a cross-section containing a right angle, and (ii) as a cross-section in which the opening defines a curve.

29. The system according to claim 28, further comprising a second device that is substantially structurally equivalent to the first device and that, in addition, contains at its arm, a data-recording unit containing an optical system with a field-of-view (FOV) defined to cover and include a tip of the nozzle when the dispenser is retained at the opening
